(19) United States
(12) Patent Application Publication
(10) Pub. No.: US 2016/0060697 A1
(43) Pub. Date: Mar. 3, 2016

(54) Compositions and Methods for Evaluating Heart Failure

(71) Applicant: LUXEMBOURG INSTITUTE OF HEALTH, Strassen (LU)

(72) Inventors: Yvan Devaux, Zoufftgen (FR); Mélanie Vausort, Tuntange (LU); Lu Zhang, Metz (FR); Daniel Wagner, Bertrange (LU); Iain Squire, Oadby, Leicestershire (GB)

(73) Assignee: LUXEMBOURG INSTITUTE OF HEALTH, Strassen (LU)

(21) Appl. No.: 14/647,680

(22) PCT Filed: Nov. 27, 2013
(86) PCT No.: PCT/EP2013/074907

§ 371 Date: May 27, 2015

Related U.S. Application Data
(60) Provisional application No. 61/730,459, filed on Nov. 27, 2012.

(30) Foreign Application Priority Data
Nov. 27, 2012 (LU) 92106

(51) Int. Cl. C12Q 1/68; G06F 19/20; C12N 15/113; G06F 19/24

(52) U.S. Cl. CPC C12Q 1/6883; G06F 19/24; G06F 19/20; C12N 15/113; C12Q 2600/178; C12Q 2600/106; C12Q 2600/118; C12N 2310/141; C12N 2320/30

(57) ABSTRACT

The present invention provides compositions and kits comprising miRNAs useful for the monitoring or diagnosis of heart disease in an individual. In particular, the compositions of the invention can be used for the prognosis of patients towards the development of left ventricular remodeling having suffered from an acute myocardial infarction. In addition, the present invention provides pharmaceutical compositions for the treatment of left ventricular remodeling.

### FIELD OF THE INVENTION

The present invention relates to compositions and kits comprising miRNAs useful for monitoring the diagnosis or progression of heart disease in an individual. In particular the compositions of the invention can be used for the prognosis of patients having suffered from an acute myocardial infarction.

### INTRODUCTION TO THE INVENTION

Heart disease encompasses a family of disorders, such as cardiomyopathies, and is a leading cause of morbidity and mortality in the industrialized world. Disorders within the heart disease spectrum are understood to arise from pathogenic changes in distinct cell types, such as cardiomyocytes, via alterations in a complex set of biochemical pathways. Left ventricular (LV) remodelling develops after acute myocardial infarction (AMI) in a significant proportion of patients¹. Associated mortality and morbidity are important and may be prevented or at least alleviated by personalized health care. To achieve this goal, however, it is critical to identify new tools to accurately predict the development of LV remodelling. Whereas N-terminal pro-brain natriuretic peptide (Nt-pro-BNP) is known to be associated with LV dysfunction after AMI, it fluctuates after AMI and better predicts poor outcome when measured 3-5 days after AMI². Talwar S et al (2000) Eur. Heart J. 21:1514-1521 showed that Nt-pro-BNP was an independent predictor of wall motion index score (WMIS), an indicator of LV contractility and remodelling.

Since the discovery of their stability in the bloodstream^{3, 4}, microRNAs (miRNAs), short oligonucleotides which down-regulate gene expression, have been the focus of a plethora of biomarker studies. Their potential to diagnose AMI has been suggested by multiple reports^{5,6}. However, their prognostic value has received much less attention, and only cardiomyocytes-enriched miRNAs have been evaluated⁷ and WO2008042231. Interestingly, the temporal profile of circulating miRNAs is related to the development of LV remodelling after AMI⁸, which suggests their usefulness as prognostic biomarkers.

Creemers, Esther E. et al: Circulation Research, Vol. 110, no. 3, February 2012 (2012-02), pages 483-495 discloses various miRNAs in connection with evaluating various cardiovascular diseases.

WO 2008/043521 discloses a large number of miRNAs, including those of the present invention, for evaluating and treating a cardiac disease.

Di Stefano, Valeria et al: Vascular Pharmacology, Vol. 55, no. 4, sp. ISS. Si, (2011-10), pages 111-118 discloses various miRNAs as markers.

WO 2008/042231 discloses a list of microRNAs, including miR-101 and miR-27a, as suitable markers for evaluating heart diseases.

### SUMMARY OF THE INVENTION

In the present invention, we have shown that a group of 4 miRNAs, miR-16 as shown in SEQ ID NO:1, miR-27a as shown in SEQ ID NO:2, miR-101, as shown in SEQ ID NO:3, miR-150 as shown in SEQ ID NO:4, (i.e. further indicated as the miRNA panel of the invention) can add to the predictive value (or prognostic value) of the existing marker, i.e. Nt-pro-BNP, in a prospective cohort of AMI patients. The potential of the miRNA panel was shown to aid in the prognostication of patients having suffered from acute myocardial infarction.

The four miRNAs of the present invention were selected from a pool of 695 possible miRNAs and, surprisingly, it has been found that only these four, in specific combination, are able to enhance the prognosis of left ventricular remodelling, preferably in combination with Nt-pro-BNP.

In a particular aspect, the present invention shows an added value of the 4 miRNA panel to Nt-pro-BNP as shown in SEQ ID NO:5, to classify patients which have suffered from myocardial infarction. In particular, the sensitivity of the prediction was improved, and the specificity was preserved.

In a particular aspect the invention provides a biomarker panel comprising miR-16, miR-27a, miR-101 and miR-150 for monitoring the prognosis of a patient having suffered from acute myocardial ischemia.

In yet another aspect the invention provides a biomarker panel comprising miR-16, miR-27a, miR-101 and miR-150 and Nt-pro-BNP for monitoring the prognosis of a patient having suffered from acute myocardial ischemia.

In yet another aspect the invention provides the use of a biomarker panel comprising miR-16, miR-27a, miR-101 and miR-150 for monitoring the prognosis of a patient having suffered from acute myocardial ischemia.

In yet another aspect the invention provides the use of a biomarker panel comprising miR-16, miR-27a, miR-101 and miR-150 and Nt-pro-BNP for monitoring the prognosis of a patient having suffered from acute myocardial ischemia.

In still another aspect the invention provides a method for predicting and/or monitoring the prognosis of a patient having suffered from an acute myocardial infarction comprising determining the levels of miR-16, miR-27a, miR-101 and miR-150 in a body fluid of said patient and correlating the levels of said miRNAs with a previously established classification model wherein said model was developed by fitting data from a study of a population of patients and said fitted data comprises levels of said biomarkers and conversion to the development of left ventricular remodeling in said selected population of patients and wherein a prognostic score is obtained for being at risk of developing left ventricular modeling.

In still another aspect the invention provides a method for predicting and/or monitoring the prognosis of a patient having suffered from an acute myocardial infarction comprising determining the levels of miR-16, miR-27a, miR-101, miR-150 and Nt-pro-BNP in a body fluid of said patient and correlating the levels of said miRNAs and Nt-pro-BNP with a previously established classification model wherein said model was developed by fitting data from a study of a population of patients and said fitted data comprises levels of said biomarkers and conversion to the development of left ventricular remodeling in said selected population of patients and wherein a prognostic score is obtained for being at risk of developing left ventricular modeling.

In particular aspect the patient having suffered from an acute myocardial infarction has a WMIS score between 1 and 1.4.

In yet another aspect the invention provides a method for assessing the efficacy of a treatment for a patient having suffered from an acute myocardial infarction and having a likelihood of developing a reduced LV contractility wherein the method comprises i) determining the levels of miR-16, miR-27a, miR-101 and miR-150 in a body fluid of said patient, ii) determining the Nt-pro-BNP level in a body fluid of said patient, iii) determining the levels of miR-16, miR-27a, miR-101 and miR-150 and the level of Nt-pro-BNP in a body fluid of said patient after treatment, iv) comparing the results of i) and ii) with the results of iii), wherein a difference between the results of i), ii) and iii) indicates an effect of the treatment.

In a particular aspect a patient has a WMIS score between 1 and 1.4.

In still other particular aspects the body fluid is blood, plasma or serum.

In yet another aspect the invention provides a diagnostic/prognostic kit for carrying out any of combination of the herein before cited methods.

In still another aspect the invention provides a composition of i) at least one short interfering nucleic acid capable of encoding a miRNA selected from the list consisting of miR-101 and miR-150 and at least one short interfering nucleic acid capable of inhibiting a miRNA selected from the list consisting of miR-16 and miR-27a or ii) short interfering nucleic acids capable of encoding miR-101 and miR-150 or iii) short interfering nucleic acids capable of inhibiting miR-16 and miR-27a for the treatment of left ventricular remodeling.

In yet another aspect the invention provides pharmaceutical formulations comprising the previous compositions.

### FIGURES

FIG. 1: Risk estimates for clinical parameters, Nt-pro-BNP and miRNAs. Nt-pro-BNP and miRNAs were measured at discharge from the hospital and LV contractility was evaluated by WMIS at 6 months follow-up. Censored regression models were built to determine the risk of impaired LV contractility. A. Model 1 is a multivariable model including indicated clinical parameters and Nt-pro-BNP. B. Model 2 is a multivariable model including the variables of model 1 and the expression values of miR-16/27a1101/150. CI: confidence interval; OR:odd ratio.

FIG. 2: Bootstrap internal validation (censored regression). Represented is the number of times that a combination of miRNAs was selected as providing the best improvement of the prediction of model 1. Data are expressed as percentage of the number of selection relative to 150 iterations.

FIG. 3: Risk estimates obtained by logistic regression. Nt-pro-BNP and miRNAs were measured at discharge and LV contractility was evaluated by WMIS at follow-up. Patients were dichotomized according to WMIS using a threshold value of 1.2. Patients with WMIS≦1.2 had preserved LV contractility (n=79) and patients with WMIS>1.2 had impaired LV contractility (n=71). Logistic regression models were built to determine the risk of impaired LV contractility. A. Model 3 is a multivariable model including indicated clinical parameters and Nt-pro-BNP. B. Model 4 is a multivariable model including the variables of model 1 and the expression values of miR-16127a1101/150. CI: confidence interval; OR:odd ratio. Note: X axis is in log scale.

FIG. 4: Bootstrap internal validation (logistic regression). Represented is the number of times that a combination of miRNAs was selected as providing the best improvement of the prediction of model 3. Data are expressed as percentage of the number of selection relative to 150 iterations.

FIG. 5 shows systems-based identification of candidate miRNAs. A. Network of interactions between proteins known to be associated with LV remodelling in humans (dark grey nodes) and 26 interacting proteins (light grey).

FIG. 6 shows expression of differentiation-related genes in early endothelial progenitor cells treated by anti-miR-16.

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 100), John Wiley & Sons, New York (2012), for definitions and terms of the art. The definitions provided herein should not be construed to have a scope less than understood by a person of ordinary skill in the art.

In the present invention we demonstrate the prognostic value of an assay comprising a panel of 4 different miRNAs in AMI patients. In particular, the combination of a panel of 4 specific miRNAs (i.e. miR-16, miR-27a, miR-101 and miR-150) and the determination of Nt-pro-BNP is found to improve the prognostic value of the gold standard Nt-pro-BNP as a stand-alone prognostic marker. The method of the invention increases the sensitivity from 48 to 60%, while maintaining the specificity at 75%. In addition, the positive predictive value was increased form 67% to 71%, and the negative predictive value was increased form 58% to 64%. One particular advantage of the invention is that the method for prognosis also improves the classification of patients with intermediate phenotypes, particularly dyskinetic patients, which are difficult to classify using existing biomarkers.

Accordingly, the invention provides in a first embodiment a biomarker panel comprising comprising miR-16, miR-27a, miR-101 and miR-150 for monitoring the prognosis of a patient having suffered from acute myocardial ischemia.

In yet another embodiment a biomarker panel is provided comprising miR-16, miR-27a, miR-101, miR-150 and Nt-pro-BNP for monitoring the prognosis of a patient having suffered from acute myocardial ischemia.

In yet another embodiment the invention provides the use of a biomarker panel comprising miR-16, miR-27a, miR-101 and miR-150 for monitoring the prognosis of a patient having suffered from acute myocardial ischemia.

In yet another embodiment the invention provides the use of a biomarker panel comprising miR-16, miR-27a, miR-101, miR-150 and Nt-pro-BNP for monitoring the prognosis of a patient having suffered from acute myocardial ischemia.

In yet another embodiment the invention provides a method for predicting and/or monitoring the prognosis of a patient having suffered from an acute myocardial infarction comprising determining the levels of miR-16, miR-27a, miR-101 and miR-150 in a body fluid of said patient and correlating the levels of said miRNAs with a previously established classification model wherein said model was developed by fitting data from a study of a population of patients and said fitted data comprises levels of said biomarkers and conversion to the development of left ventricular remodeling in said selected population of patients and wherein a prognostic score is obtained for being at risk of developing left ventricular modeling.

In yet another embodiment the invention provides a method for predicting and/or monitoring the prognosis of a patient having suffered from an acute myocardial infarction comprising determining the levels of miR-16, miR-27a, miR-101 and miR-150 in a body fluid of said patient and correlating the levels of said miRNAs with a previously established classification model wherein said model was developed by fitting data from a study of a population of patients and said fitted data comprises levels of said biomarkers and conversion to the development of left ventricular remodeling in said selected population of patients and wherein a prognostic score is obtained for being at risk of developing left ventricular modeling and wherein a practitioner starts a treatment plan based on the prognostic score.

In a particular embodiment the treatment plan involves the administration of a drug, such as an ACE inhibitor, an angiotensin H receptor blocker, a Beta-blocker, a vasodilator, a pro-angiogenic factor, a cardiac glycoside, an antiarrhythmic agent, a diuretic, a statin, or an anticoagulant, an inotropic agent; an immunosuppressive agent, use of a pacemaker, defibrillator, mechanical circulatory support, surgery, or therapy with stem cells (bone marrow derived stem cells, mesenchymal stem cells, cardiac stem cells, muscle derived stem cells).

The wording “for being at risk of developing left ventricular modeling” is equivalent to the wording “for being at risk of developing a reduced left ventricular contractility”.

In yet another embodiment the invention provides a method for predicting and/or monitoring the prognosis of a patient having suffered from an acute myocardial infarction comprising: i) determining the levels of miR-16, miR-27a, miR-101 and miR-150 in a body fluid of said patient, ii) determining the Nt-pro-BNP level in a body fluid of said patient wherein the levels of said miRNAs and said Nt-pro-BNP level is correlated with a previously established classification model wherein said model was developed by fitting data from a study of a population of patients and said fitted data comprises levels of said biomarkers and conversion to the development of left ventricular remodeling in said selected population of patients and wherein a prognostic score is obtained for being at risk of developing left ventricular modeling.

In a particular embodiment the body fluid for measuring the levels of Nt-pro-BNP and the body fluid for measuring the levels of miR-16, miR-27a, miR-101 and miR-150 are different body fluids.

In a particular embodiment a body fluid is blood, serum, plasma, Cerebro Spinal Fluid (CSF), saliva or urine.

In a preferred embodiment the body fluid is blood, serum or plasma.

In particular embodiments the body fluid of a patient having suffered from an acute myocardial infarction is sampled after 5 minutes, 10 minutes, 60 minutes, 2 hours, 12 hours, 1 day, 2 days, 3 days, 4 days, 5 days or after even a longer period. In a particular embodiment the body fluid is sampled at any time point between 5 minutes and 4 weeks after the acute myocardial infarction.

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70° C. until thawed and used. “Fresh” plasma or serum should be refrigerated or maintained on ice until used, with nucleic acid extraction being performed as soon as possible. Blood can be drawn by standard methods into a collection tube, typically siliconized glass, either without anticoagulant for preparation of serum, or with EDTA, sodium citrate, heparin, or similar anticoagulants for preparation of plasma. When preparing plasma or serum for storage, although not an absolute requirement, is that plasma or serum is first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using gentle centrifugation at 300-800 times gravity for five to ten minutes, or fractionated by other standard methods. High centrifugation rates capable of fractionating out apoptotic bodies should be avoided. Since heparin may interfere with RT-PCR, use of heparinized blood may require pretreatment with heparanase, followed by removal of calcium prior to reverse transcription. Imai, H. et al (1992) J. Virol. Methods 36:181-184.

An AMI patient is a patient who has suffered from an acute myocardial infarction.

In the present invention the classification model is established with patients who have suffered from an acute myocardial infarction. Typically, for establishing the model patients are recruited who developed left ventricular remodeling and patients who did not develop left ventricular remodeling.

The wording “a method for predicting and/or monitoring the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given characteristic, such as the presence or level of a prognostic indicator, when compared to those individuals not exhibiting the characteristic. For example, as described hereinafter, an AMI patient exhibiting a high level of miR-16 and mi-R27a and a low level of miR-150 and miR-101 and an increased level of Nt-pro-BNP, as compared to a mean value determined in a population of patients included in the classification model, may be more likely to suffer or to progress towards a patient with an impaired LV contractility. In preferred embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

The skilled artisan will understand that associating a prognostic indicator with a predisposition to an outcome of reduced LV contractility is a statistical analysis. For example, changes in the miRNA panel as described herein in combination with a change in the amount of Nt-pro-BNP may signal that a patient, in particular an AMI patient, is more likely to suffer from an adverse outcome than patients with different levels, as determined by a level of statistical significance. Common tests for evaluating statistical significance include but are not limited to ANOVA, Kniskal-Wallis, t-test and odds ratio (OR). Statistical significance is often determined by comparing two or more populations, and determining a confidence interval (CI) and/or a p value. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described hereinafter.

The term “correlating,” as used herein in reference to the use of prognostic indicators to determine a prognosis, refers to comparing the presence or amount of the prognostic indicator in a patient to its presence or amount in persons known to suffer from, or known to be at risk of, a given condition; or in persons known to be free of a given condition. For example, the miRNA panel levels in a patient can be compared to a level known to be associated with an increased disposition of developing an impaired left ventricular contractility. The patient's miRNA panel levels are said to have been correlated with a prognosis; that is, the skilled artisan can use the patient's miRNA panel levels, optionally in combination with the determination of the Nt-pro-BNP levels, to determine the likelihood that the patient is at risk for developing impaired LV contractility or dyskinesia, and respond accordingly. Alternatively, the patient's miRNA panel levels can be compared to a miRNA panel level known to be associated with a good outcome (e.g., no impaired LV contractility, no risk for sudden death, etc.), and determine if the patient's prognosis is predisposed to the good outcome.

As used herein, expression pattern refers to the combination of occurrences or levels in a set of miRNAs of a sample. In assessing the similarity of two expression patterns, for example, a test expression pattern and a reference expression pattern, a comparison is made between the occurrences or levels of the same miRNAs in the test and reference (or control) expression patterns for each of the four miRNA pairs. In one embodiment the classification scheme involves building or constructing a statistical model also referred to as a classifier or predictor, that can be used to classify samples to be tested (test samples) based on miRNA levels or occurrences. The model is built using reference samples (control samples) for which the classification has already been ascertained, referred to herein as a reference dataset comprising reference expression patterns. Hence, reference expression patterns are levels or occurrences of a set of one or more miRNAs in a reference sample (e.g. a reference blood or plasma or serum sample). Once the model (classifier) is built, then a test expression pattern obtained from a test sample is evaluated against the model (e.g. classified as a function of relative miRNAs expression of the sample with respect to that of the model). In some embodiments, evaluation involves identifying the reference expression pattern that most closely resembles the expression pattern of the test sample and associating the known reduced left ventricular contractility class or type of the reference expression pattern with the test expression pattern, thereby classify (categorizing) the risk towards developing a reduced left ventricular contractility associated with the test expression pattern. The number of relevant miRNAs to be used for building the model can be determined by one of skill in the art. In one embodiment, a greedy search method (backward selection) with Support Vector Machine is used to determine a subset of miRNAs that can be chosen to build a model (e.g., Naive Bayes and Logistic regression) for prediction of the presence of left ventricular contractility reduction. A class prediction strength can also be measured to determine the degree of confidence with which the model classifies a sample to be tested. The prediction strength conveys the degree of confidence of the classification of the sample and evaluates when a sample cannot be classified. There may be instances in which a sample is tested, but does not belong to a particular class. This is done by utilizing a threshold wherein a sample which scores below the determined threshold is not a sample that can be classified (e.g., a “no call”). The prediction strength threshold can be determined by the skilled artisan based on known factors, including, but not limited to the value of a false positive classification versus a “no call.” Once a model is built, the validity of the model can be tested using methods known in the art. One way to test the validity of the model is by cross-validation of the dataset. To perform cross-validation, one of the samples is eliminated and the model is built, as described above, without the eliminated sample, forming a “cross-validation model.” The eliminated sample is then classified according to the model, as described herein. This process is done with all the samples of the initial dataset and an error rate is determined. The accuracy of the model is then assessed. This model classifies samples to be tested with high accuracy for classes that are known, or classes that have been previously ascertained or established through class discovery as discussed herein. Another way to validate the model is to apply the model to an independent data set, such as a new unknown test plasma or blood or serum sample. Other standard biological or medical research techniques, known or developed in the future, can be used to validate class discovery or class prediction.

Classification of the sample gives a healthcare provider information about a classification to which the sample belongs, based on the analysis of the levels of the miRNA panel of the invention, optionally including the determination of Nt-pro-BNP levels. The information provided by the present invention, alone or in conjunction with other test results, aids the healthcare provider in diagnosing the individual. Also, the present invention provides methods for determining a treatment plan. Once the health care provider knows to which disease class (i.e. being at risk for developing LV remodeling or not) the sample, and therefore, the individual belongs, the health care provider can determine an adequate treatment plan for the individual. For example, different assessments of left ventricular contractility reduction often require differing treatments. Properly diagnosing and understanding the seriousness of left ventricular remodeling of an individual allows for a better, more successful treatment and prognosis. Other applications of the invention include classifying persons who are likely to have successful treatment with a particular drug or therapeutic regiment. Those interested in determining the efficacy of a drug for reducing left ventricular remodeling can utilize the methods of the present invention.

In yet another embodiment the invention relates to a method of assessing the efficacy of a treatment for a patient having suffered from an acute myocardial infarction and is at risk for developing a reduced LV contractility wherein the method comprises i) determining the levels of miR-16, miR-27a, miR-101 and miR-150 in a body fluid of said patient, ii) determining the Nt-pro-BNP level in a body fluid of said patient, iii) determining the levels of miR-16, miR-27a, miR-101 and miR-150 and the level of Nt-pro-BNP in a body fluid of said patient after treatment, iv) comparing the results of i) and ii) with the results of iii), wherein a difference between the results of i), ii) and iii) indicates an effect of the treatment.

In certain embodiments, the treatment is the administration of a drug, such as an ACE inhibitor, an angiotensin II receptor blocker, a Beta-blocker, a vasodilator, a cardiac glycoside, an antiarrhythmic agent, a diuretic, statins, or an anticoagulant, an inotropic agent; an immunosuppressive agent, use of a pacemaker, defibrillator, mechanical circulatory support, or surgery.

Assay measurement strategies: numerous methods and devices are well known to the skilled artisan for measuring the prognostic indicators of the instant invention. With regard to polypeptides, such as Nt-pro-BNP, in patient samples, immunoassay devices and methods are often used. See, e.g. U.S. Pat. No. 6,143,576, U.S. Pat. No. 6,113,855 and U.S. Pat. No. 6,019,944. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g. U.S. Pat. No. 5,631,171 and U.S. Pat. No. 5,955,377.

With regard to the determination of the 4 miRNAs of the invention, the expression of these 4 miRNAs can be measured separately or simultaneously. The miRNA expression levels are obtained, e.g. by using a quantitative RT-PCR or a bead-based system. In a particular embodiment a suitable array-based system (e.g. miRMAX microarray, GeneXpert System Cepheid, MDx platform Biocartis) can be developed and the extent of hybridization of the miRNAs in the sample to the beads or the probes on the microarray is determined. Once the miRNA expression levels of the sample are obtained, the levels are compared or evaluated against the model and the patient sample is classified.

In another particular embodiment the levels of miR-150 and miR101 in the body fluid derived from a patient having suffered from an AMI and being at risk for developing an impaired LV contractility are lower than the corresponding miRNA levels in the corresponding body fluid of a group of control patients. A control patient is typically a patient having suffered from an AMI and having preserved LV contractility. In a particular embodiment a control patient is a patient who has not suffered from an AMI and is also an individual with a preserved LV contractility. In a particular embodiment the levels of miR-150 and miR101 in the body fluid derived from a patient having suffered from an AMI and being at risk for developing an impaired LV contractility are at least 2-fold lower, at least 3-fold lower, at least 4-fold lower, at least 5-fold lower than the levels of the corresponding miRNA levels in the corresponding body fluid of a control patient.

In yet another particular embodiment the levels of miR-16 and miR27a in the body fluid derived from a patient having suffered from an AMI and being at risk for developing an impaired LV contractility are higher than the corresponding miRNA levels in the corresponding body fluid of a control patient. In a particular embodiment the levels of miR-16 and miR27a in the body fluid derived from a patient having suffered from an AMI and being at risk for developing an impaired LV contractility are at least 2-fold higher, at least 3-fold higher, at least 4-fold higher, at least 5-fold higher than the levels of the corresponding miRNA levels in the corresponding body fluid of a control patient.

In yet another embodiment for each increase of 1 unit of miR-16 in the patient body fluid the likelihood of classifying said patient into the category (or class) of developing a reduced left ventricular contractility is increased by 4.2 fold and for each increase of 1 unit of miR-27a in the patient body fluid the likelihood of classifying said patient into the category of developing a reduced left ventricular contractility is increased by 15.9 fold and for each increase of 1 unit of miR-101 in a patient body fluid the likelihood of classifying said patient into the high risk category of developing a reduced left ventricular contractility is decreased by 5.2 fold and for each increase of 1 unit of miR-150 in a patient body fluid, the likelihood of classifying said patient into the high risk category of developing a reduced left ventricular contractility is decreased by 12.1 fold and for an at least 3-fold increase of Nt-pro-BNP in a patient body fluid there is a high likelihood that a patient will develop a reduced left ventricular contractility.

In yet another embodiment the invention provides a kit for determining the prognosis of a patient diagnosed with an acute myocardial infarction. These kits preferably comprise devices and reagents for measuring the Nt-pro-BNP level in a patient sample, and devices and reagents for measuring the panel of 4 miRNAs of the invention and instructions for performing the assays. Optionally, the kits may contain one or more means for converting the Nt-pro-BNP levels and miRNA panel levels to a prognosis.

In yet another embodiment the invention relates to methods for the treatment of left ventricular modeling. In a specific embodiment the treatment is conditional to the value of the prognostic score obtained through the method for predicting and/or monitoring the prognosis of a patient having suffered from an acute myocardial infarction of the present invention.

Thus the present invention relates to methods useful for the treatment of left ventricular remodeling (or reduced left ventricular cardiac contractility) based on the supplementation of miR-150 and/or miR-101 and/or in combination with the inhibition of miR-16 and/or miR-27a.

In yet another particular embodiment the invention provides a composition of
i) at least one short interfering nucleic acid capable of encoding a miRNA selected from the list consisting of miR-101 and miR-150 and at least one short interfering nucleic acid capable of inhibiting a miRNA selected from the list consisting of miR-16 and miR-27a or
ii) ii) short interfering nucleic acids capable of encoding miR-101 and miR-150 or
iii) iii) short interfering nucleic acids capable of inhibiting miR-16 and miR-27a for the treatment of left ventricular remodeling.

The wording ‘at least one short interfering nucleic acid capable of encoding a miRNA selected from the list consisting of miR-101 and miR-150’ refers to the supplementation of the miRNA expression of miR-150 or miR-101 which is downregulated in patients predicted with a prognosis of developing cardiac left ventricular remodeling. A short interfering nucleic acid capable of encoding a miRNA can for example be a microRNA, a short interfering RNA, a double-stranded RNA or a short hairpin RNA. A short interfering nucleic acid of the present invention can be chemically synthesized, expressed from a vector or enzymatically synthesized. The use of chemically-modified short interfering nucleic acids improves various properties of native short interfering nucleic acid molecules through, for example, increased resistance to nuclease degradation in vivo and/or through improved cellular uptake. Chemically synthesizing nucleic acid molecules with modifications (base, sugar and/or phosphate) that prevent their degradation by serum ribonucleases can increase their potency. There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups. In one embodiment the short interfering nucleic acid molecule is double stranded and each strand of the short interfering nucleic acid molecule comprises about 19 to about 23 nucleotides, and each strand comprises at least about 19 nucleotides that are complementary to the nucleotides of the other strand.

In yet another embodiment, each strand of the short interfering nucleic acids comprises about 16 to about 25 nucleotides.

In some embodiments, a short interfering nucleic acid sequence is substantially similar to the sequence of the selected miRNA, or is a short interfering nucleic acid sequence which is identical to the selected miRNA sequence at all but 1, 2, 3, 4, 5, 6, 7, 8, 9, 10, 11, 12, 13, 14, 15, 16, 17, or 18 bases. In some embodiments, the short interfering nucleic acid sequence is a sequence that is substantially similar to the sequence of an miRNA, or is a short interfering nucleic acid sequence that is different than the miRNA sequence at all but up to one base. In some embodiments, a miRNA is supplemented by delivering an siRNA having a sequence that comprises the sequence, or a substantially similar sequence, of the miRNA. In still other embodiments, miRNAs are supplemented by delivering miRNAs encoded by shRNA vectors. Such technologies for delivery exogenous microRNAs to cells are well known in the art.

The wording “a short interfering nucleic acid capable of inhibiting a miRNA” refers to the inhibition of a selected miRNA function. A miRNA in itself inhibits the function of the mRNAs it targets and, as a result, inhibits expression of the polypeptides encoded by the mRNAs. Thus, blocking (partially or totally) or inhibiting the activity of a selected miRNA can effectively induce, or restore, expression of a polypeptide whose expression is inhibited (derepress the polypeptide). In one embodiment, derepression of polypeptides encoded by mRNA targets of a selected miRNA is accomplished by inhibiting the miRNA activity in cells through any one of a variety of methods. For example, blocking the activity of a miRNA can be accomplished by hybridization with a short interfering nucleic acid that is complementary, or substantially complementary to, the miRNA, thereby blocking interaction of the miRNA with its target mRNA. As used herein, a short interfering nucleic acid that is substantially complementary to a miRNA is a short interfering nucleic acid that is capable of hybridizing with a selected miRNA, thereby blocking the miRNA's activity. In some embodiments, a short interfering nucleic acid that is substantially complementary to a miRNA is a short interfering nucleic acid that is complementary with the miRNA at all but 1, 2, 3, 4, 5, 6, 7, 8, 9, 10, 11, 12, 13, 14, 15, 16, 17, or 18 bases. In some embodiments, a short interfering nucleic acid sequence is a sequence that is substantially complementary to a miRNA, or is a short interfering nucleic acid sequence that is complementary with the miRNA at, at least, one base. In particular embodiments antisense oligonucleotides, including chemically modified antisense oligonucleotides—such as 2′ O-methyl, locked nucleic acid (LNA)—inhibit miRNA activity by hybridization with guide strands of mature miRNAs, thereby blocking their interactions with target mRNAs. In further particular embodiments ‘antagomirs’ are phosphorothioate modified oligonucleotides that can specifically block a selected miRNA in vivo (see for example Kurtzfeldt, J. et al. (2005) Nature 438, 685-689). In still other particular embodiments microRNA inhibitors, termed miRNA sponges, can be expressed in cells from transgenes (see for example Ebert, M. S. (2007) Nature Methods, 12). These miRNA sponges specifically inhibit selected miRNAs through a complementary heptameric seed sequence and even an entire family of miRNAs can be silenced using a single sponge sequence. Other methods for silencing miRNA function in cells will be apparent to one of ordinary skill in the art.

In yet another embodiment the invention contemplates the use of the compositions as described herein before for the treatment of a human subject which may be a pediatric, an adult or a geriatric subject, wherein said human subject is predicted to develop left ventricular remodeling. As used herein treatment, or treating, includes amelioration, cure of a left ventricular remodeling. In specific embodiments the invention provides a pharmaceutical pack or kit comprising one or more containers filled with one or more of the ingredients of the pharmaceutical compositions of the invention. Associated with such container(s) can be various written materials (written information) such as instructions (indicia) for use, or a notice in the form prescribed by a governmental agency regulating the manufacture, use or sale of pharmaceuticals or biological products, which notice reflects approval by the agency of manufacture, use or sale for human administration. The pharmaceutical compositions of the present invention preferably contain a pharmaceutically acceptable carrier or excipient suitable for rendering the compound or mixture administrable orally as a tablet, capsule or pill, or parenterally, intravenously, intradermally, intramuscularly or subcutaneously, or transdermally. The active ingredients may be admixed or compounded with any conventional, pharmaceutically acceptable carrier or excipient. As used herein, the term “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention, its use in the therapeutic formulation is contemplated. Supplementary active ingredients can also be incorporated into the pharmaceutical formulations. A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 21^{st} ed. (2012), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention. An effective amount, also referred to as a therapeutically effective amount, of a short interfering nucleic acid as described herein before is an amount sufficient to ameliorate at least one adverse effect associated with expression, or reduced expression, of the selected microRNA in a cell (for example a myocardial cell) or in an individual in need of such inhibition or supplementation. The therapeutically effective amount of the short interfering nucleic acid (active agent) to be included in pharmaceutical compositions depends, in each case, upon several factors, e.g. the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg. One of ordinary skill in the art would be able to determine empirically an appropriate therapeutically effective amount. Use of the small interfering nucleic acid-based molecules of the invention can lead to better treatment of the disease progression by affording, for example, the possibility of combination therapies with known drugs, or intermittent treatment with combinations of small interfering nucleic acids and/or other chemical or biological molecules). In some embodiments therapeutic short interfering nucleic acids of the invention delivered exogenously are optimally stable within cells until translation of the target mRNA has been inhibited long enough to reduce the levels of the protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above. The administration of the herein described small interfering nucleic acid molecules to a patient can be intravenous, intraarterial, intraperitoneal, intramuscular, subcutaneous, intrapleural, intrathecal, by perfusion through a regional catheter, or by direct intralesional injection. When administering these small interfering nucleic acid molecules by injection, the administration may be by continuous infusion, or by single or multiple boluses. The dosage of the administered nucleic acid molecule will vary depending upon such factors as the patient's age, weight, sex, general medical condition, and previous medical history. Typically, it is desirable to provide the recipient with a dosage of the molecule which is in the range of from about 1 pg/kg to 10 mg/kg (amount of agent/body weight of patient), although a lower or higher dosage may also be administered. In some embodiments, it may be desirable to target delivery of a therapeutic to the heart, while limiting delivery of the therapeutic to other organs. This may be accomplished by any one of a number of methods known in the art. In one embodiment delivery to the heart of a pharmaceutical formulation described herein comprises coronary artery infusion. In certain embodiments coronary artery infusion involves inserting a catheter through the femoral artery and passing the catheter through the aorta to the beginning of the coronary artery. In yet another embodiment, targeted delivery of a therapeutic to the heart involves using antibody-protamine fusion proteins, such as those previously described (Song E et al. (2005) Nature Biotechnology Vol. 23(6), 709-717) to deliver the small interfering nucleic acids disclosed herein. While it is possible for the agents to be administered as the raw substances, it is preferable, in view of their potency, to present them as a pharmaceutical formulation. The formulations of the present invention for human use comprise the agent, together with one or more acceptable carriers therefor and optionally other therapeutic ingredients. The carrier(s) must be “acceptable” in the sense of being compatible with the other ingredients of the formulation and not deleterious to the recipient thereof or deleterious to the inhibitory function of the active agent. Desirably, the formulations should not include oxidizing agents and other substances with which the agents are known to be incompatible. The formulations may conveniently be presented in unit dosage form and may be prepared by any of the methods well known in the art of pharmacy. All methods include the step of bringing into association the agent with the carrier, which constitutes one or more accessory ingredients. In general, the formulations are prepared by uniformly and intimately bringing into association the agent with the carrier(s) and then, if necessary, dividing the product into unit dosages thereof. Formulations suitable for parenteral administration conveniently comprise sterile aqueous preparations of the agents, which are preferably isotonic with the blood of the recipient. Suitable such carrier solutions include phosphate buffered saline, saline, water, lactated ringers or dextrose (5% in water). Such formulations may be conveniently prepared by admixing the agent with water to produce a solution or suspension, which is filled into a sterile container and sealed against bacterial contamination. Preferably, sterile materials are used under aseptic manufacturing conditions to avoid the need for terminal sterilization. Such formulations may optionally contain one or more additional ingredients among which may be mentioned preservatives, such as methyl hydroxybenzoate, chlorocresol, metacresol, phenol and benzalkonium chloride. Such materials are of special value when the formulations are presented in multidose containers. Buffers may also be included to provide a suitable pH value for the formulation. Suitable such materials include sodium phosphate and acetate. Sodium chloride or glycerin may be used to render a formulation isotonic with the blood. If desired, the formulation may be filled into the containers under an inert atmosphere such as nitrogen or may contain an antioxidant, and are conveniently presented in unit dose or multi-dose form, for example, in a sealed ampoule.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1

### 1. Patient Characteristics

Table 1 shows the demographic features of patients of the studied population. Among the 150 patients enrolled 79 patients evidenced a loss of LV contractility at follow-up (WMIS 1.2) and 71 patients had a preserved LV contractility. Comparisons between these 2 groups of patients revealed that patients with impaired contractility had higher levels of troponin I, creatine kinase and Nt-pro-BNP at discharge than patients with preserved contractility. Diuretics were more often prescribed in these patients, and they had a higher risk of developing chronic heart failure.

TABLE 1
Demographic and clinical features of AMI patients

| | All | | WMIS ≦ 1.2 | | WMIS > 1.2 | | |
|---|---|---|---|---|---|---|---|
| | (N = 150) | | (N = 79) | | (N = 71) | | P¹ |
| Age, y (median-range) | 64 | (24-87) | 61 | (37-86) | 65 | (24-87) | 0.56 |
| Male, n (%) | 116 | (77%) | 63 | (80%) | 53 | (75%) | 0.89 |
| Cardiovascular history/ | | | | | | | |
| risk factors, n (%) | | | | | | | |
| Smoker | 60 | (40%) | 33 | (42%) | 27 | (38%) | 0.88 |
| FH | 59 | (39%) | 31 | (42%) | 28 | (35%) | 0.89 |
| Angina | 14 | (28%) | 5 | (6%) | 9 | (13%) | 0.35 |
| Diabetes | 24 | (16%) | 12 | (15%) | 12 | (17%) | 1 |
| Hypertension | 52 | (35%) | 26 | (33%) | 26 | (37%) | 1 |
| Hypercholesterolaemia | 40 | (27%) | 18 | (23%) | 22 | (31%) | 0.49 |
| MI | 12 | (8%) | 3 | (4%) | 9 | (13%) | 0.12 |
| PCI | 3 | (2%) | 3 | (4%) | 0 | (0%) | 0.30 |
| CABG | 1 | (1%) | 0 | (0%) | 1 | (1%) | 0.96 |
| Presentation, n (%) | | | | | | | |
| STEMI | 127 | (85%) | 62 | (78%) | 65 | (92%) | 0.60 |
| Anterior infarct | 59 | (39%) | 24 | (30%) | 35 | (49%) | 0.16 |
| Thrombolysis | 75 | (50%) | 42 | (53%) | 33 | (46%) | 0.74 |
| Serum markers during | | | | | | | |
| admission | | | | | | | |
| (median-range) | | | | | | | |
| Troponin I (ng/mL) | 9.83 | (0.08-150) | 5.90 | (0.08-150) | 19.95 | (0.09-150) | 0.001 |
| CK (units/L) | 985 | (56-7384) | 625 | (56-3925) | 1614 | (123-7384) | <0.001 |
| Nt-pro-BNP (ng/L) | 2.80 | (0.26-3.98) | 2.53 | (0.26-3.55) | 3.16 | (0.94-3.98) | <0.001 |
| Medications at | | | | | | | |
| admission, n (%) | | | | | | | |
| Aspirin | 21 | (14%) | 9 | (11%) | 12 | (17%) | 0.54 |
| Clopidogrel | 4 | (3%) | 3 | (4%) | 1 | (1%) | 0.71 |
| Beta-blockers | 24 | (16%) | 13 | (16%) | 11 | (15%) | 0.93 |
| Calcium antagonists | 22 | (15%) | 7 | (9%) | 15 | (21%) | 0.11 |
| ACE inhibitors | 17 | (11%) | 6 | (8%) | 11 | (15%) | 0.27 |
| Angiotensin receptor | 9 | (6%) | 6 | (8%) | 3 | (4%) | 0.64 |
| blocker | | | | | | | |
| Statins | 28 | (19%) | 13 | (16%) | 15 | (21%) | 0.69 |
| Medications at | | | | | | | |
| discharge, n (%) | | | | | | | |
| Aspirin | 134 | (89%) | 73 | (92%) | 61 | (86%) | 0.85 |
| Clopidogrel | 36 | (24%) | 23 | (29%) | 13 | (18%) | 0.30 |
| Beta-blocker | 142 | 95%) | 75 | (95%) | 67 | (94%) | 0.93 |
| ACE inhibitor | 134 | (89%) | 71 | (90%) | 63 | (89%) | 0.95 |
| Angiotensin receptor | 11 | (7%) | 5 | (6%) | 6 | (8%) | 0.88 |
| blocker | | | | | | | |
| Diuretic | 15 | (10%) | 2 | (3%) | 13 | (18%) | 0.008 |
| Statin | 148 | (99%) | 78 | (99%) | 70 | (99%) | 0.91 |
| Endpoints at 6-months | | | | | | | |
| Reinfarction, n (%) | 15 | (10%) | 5 | (6%) | 10 | (14%) | 0.25 |
| CHF, n (%) | 11 | (7%) | 1 | (1%) | 10 | (14%) | 0.01 |
| Death, n (%) | 4 | (3%) | 1 | (1%) | 3 | (4%) | 0.56 |

¹For comparison between WMIS ≦ 1.2 and WMIS > 1.2.
ACE: angiotensin-converting enzyme; BNP: brain natriuretic peptide; CABG: coronary artery bypass grafting; CHF: congestive heart failure; CK: creatine kinase; FH: familial hypercholesterolemia; MI: myocardial infarction; PCI: percutaneous coronary intervention; STEMI: ST-elevation myocardial infarction.

Table 2 shows the parameters of LV function as assessed by echocardiography, at discharge from the hospital and at 6-months follow-up. Patients who subsequently developed a loss of LV contractility had lower EF and higher LV volumes and diameters compared to patients with preserved LV contractility, at discharge from the hospital as well as after 6 months.

TABLE 2
Echo parameters of AMI patients

| | All | | WMIS ≦ 1.2 | | WMIS > 1.2 | | |
|---|---|---|---|---|---|---|---|
| | (N = 150) | | (N = 79) | | (N = 71) | | P¹ |
| Pre-discharge | | | | | | | |
| echo/MRI | | | | | | | |
| (median-range) | | | | | | | |
| LVEF (%) | 44 | (15-75) | 50 | (22-75) | 37 | (15-61) | <0.001 |
| LVEDV (mL) | 89 | (36-201) | 83 | (36-159) | 94 | (36-201) | 0.005 |
| LVESV (mL) | 46 | (20-132) | 39 | (21-86) | 56 | (20-132) | <0.001 |
| LVIDd (mm) | 4.8 | (2.9-6.6) | 4.6 | (2.9-6) | 5.2 | (3.7-8.1) | 0.006 |
| LVIDs (mm) | 3.6 | (1.8-5.5) | 3.35 | (1.8-5.2) | 4.1 | (1.7-7) | <0.001 |
| WMIS | 1.31 | (1-2.38) | 1.06 | (1-1.81) | 1.74 | (1-2.38) | <0.001 |
| Follow-up echo/MRI | | | | | | | |
| (median-range) | | | | | | | |
| LVEF (%) | 48 | (16-74) | 52 | (37-74) | 40 | (16-69) | <0.001 |
| LVEDV (mL) | 87 | (40-208) | 80 | (45-163) | 96 | (40-208) | <0.001 |
| LVESV (mL) | 45 | (17-141) | 38 | (17-89) | 58 | (17-141) | <0.001 |
| LVIDd (mm) | 4.9 | (3-8.1) | 4.8 | (3-6.3) | 5.2 | (3.7-8.1) | <0.001 |
| LVIDs (mm) | 3.6 | (1.6-7) | 3.4 | (1.6-4.9) | 4.1 | (1.7-7) | <0.001 |
| WMIS | 1.19 | (1-2.38) | 1 | (1-1.2) | 1.5 | (1.25-2.38) | <0.001 |

¹For comparison between WMIS ≦ 1.2 and WMIS > 1.2.
LVEDV: end-diastolic volume; LVESV: end-systolic volume; LVEF: ejection fraction; LVIDd: end-diastolic diameter; LVIDs: end-systolic diameter.

### 2. Estimation of Risk of Impaired LV Contractility

Levels of miR-16/27a/101/150 were measured in blood samples obtained at discharge from the hospital. Nt-pro-BNP levels at discharge were also determined in each patient. LV contractility at follow-up was assessed by echocardiographic determination of WMIS. High WMIS indicates an impairment of LV contractility. 55 patients had a WMIS equal to 1, indicating a fully preserved contractility in these patients. Due to this left censoring of WMIS values at 1, censored regression (aka “Tobit regression”) was used for prediction analysis. To compare the predictive value of miRNAs over classical markers, 2 multivariable models were built. The first model (=model 1) included the following parameters: age, gender, smoking habit, diabetes, hypertension, hypercholesterolemia, antecedent of MI, infarct type (STEMI vs NSTEMI), infarct territory (anterior vs inferior), and Nt-pro-BNP level at discharge. The second model (=model 2) included all the parameters of model 1 and the expression values of a panel of 4 miRNAs, miR-16/27a/101/150, measured in plasma samples obtained at discharge.

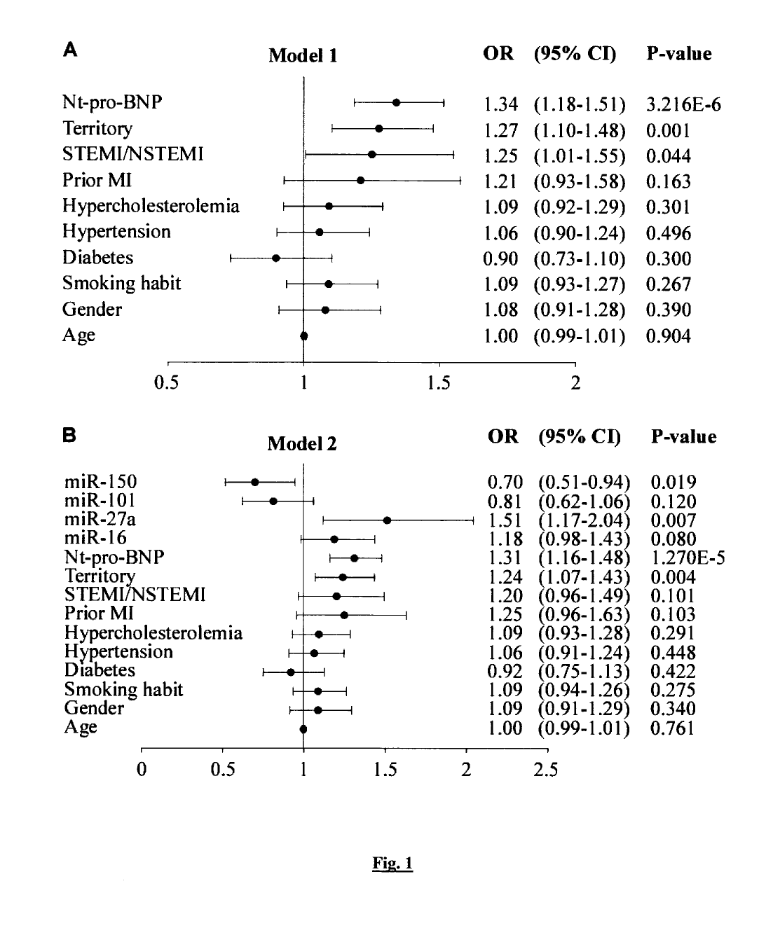

FIG. 1A shows the odds ratios (OR) of each variable in model 1. Infarct type, infarct territory and Nt-pro-BNP were significant predictors of WMIS. Patients with anterior STEMI and elevated Nt-pro-BNP were at higher risk of impaired contractility. FIG. 1B shows that miR-27a and miR-150 significantly contributed to the predictive value of model 2. The predictive values of miR-16 and miR-101 were of borderline significance.

### 3. Added Value of Combinations of miRNAs

We determined the ability of each miRNA and of combinations of several miRNAs to improve the predictive value of model 1 (Table 3). The AIC was used in this analysis since this criteria is adjusted by the number of variables, contrarily to AUC which is biased by variable number. This allows avoiding the chance of having a better prediction only by increasing the number of variables included in the model. Low AIC is indicative of accurate prediction. No single miRNA was able to add to the predictive value of model 1. All 4 miRNAs were necessary to significantly improve the model.

TABLE 3
Added value of combinations of miRNAs (censored regression)

| | Wald chi square | | LRT |
|---|---|---|---|
| miRNA added to model 1 | test P-value | AIC | P-value |
| None | 4.35E−07 | 205.386 | |
| miR-16 | 8.47E−07 | 206.853 | 0.465 |
| miR-27a | 2.66E−07 | 204.351 | 0.081 |
| miR-101 | 7.38E−07 | 206.655 | 0.392 |
| miR-150 | 9.69E−07 | 207.332 | 0.817 |
| miR-16 + miR-27a | 6.02E−07 | 206.35 | 0.219 |
| miR-16 + miR-101 | 1.61E−06 | 208.536 | 0.654 |
| miR-16 + miR-150 | 1.10E−06 | 207.643 | 0.418 |
| miR-27a + miR-101 | 3.82E−07 | 205.303 | 0.130 |
| miR-27a + miR-150 | 1.68E−07 | 203.816 | 0.062 |
| miR-101 + miR-150 | 1.20E−06 | 208.074 | 0.519 |
| miR-16 + miR-27a + miR-101 | 8.31E−07 | 207.232 | 0.245 |
| miR-16 + miR-27a + miR-150 | 1.98E−07 | 204.178 | 0.066 |
| miR-16 + miR-101 + miR-150 | 1.72E−06 | 208.95 | 0.487 |
| miR-27a + miR-101 + miR-150 | 2.49E−07 | 204.826 | 0.087 |
| miR-16 + miR-27a + miR-101 + | 1.51E−07 | 203.752 | 0.047 |
| miR-150 | | | |

Shown are the results of all combinations of miRNAs added to model 1. The Wald chi square test indicates the significance of the model. The likelihood ratio test (LRT) compares the predictive value of a model with miRNAs to model 1. AIC: Akaike information criteria.

### 4. Model Validation

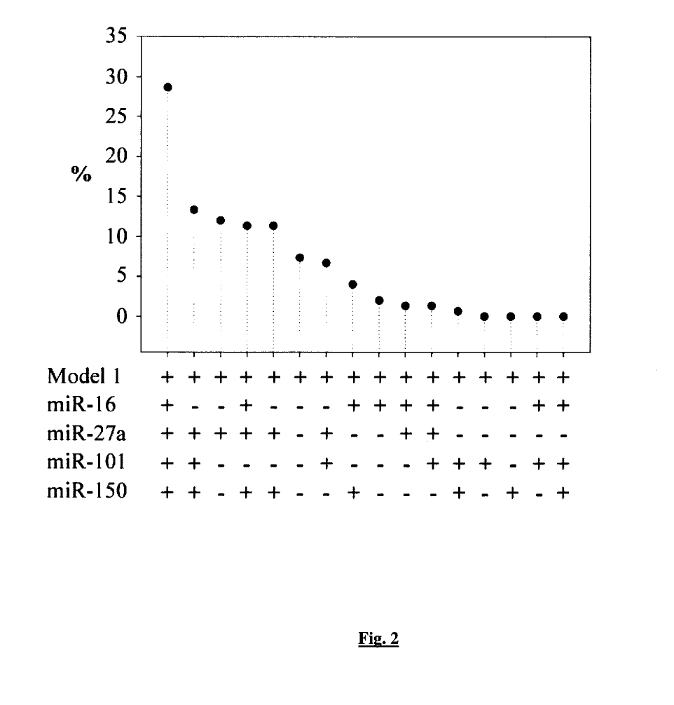

Bootstrap internal validation was used to test the strength of the models with combinations of miRNAs (FIG. 2). The principle of this test is to calculate the predictive value of the model after resampling patient from the original sample. The model including the 4 miRNAs was the best predictor in 29% of the 150 iterations performed. MiR-27a was selected in 13% of cases and was included in all top models, showing its significant contribution to the prediction.

### 5. Logistic Regression Analyses

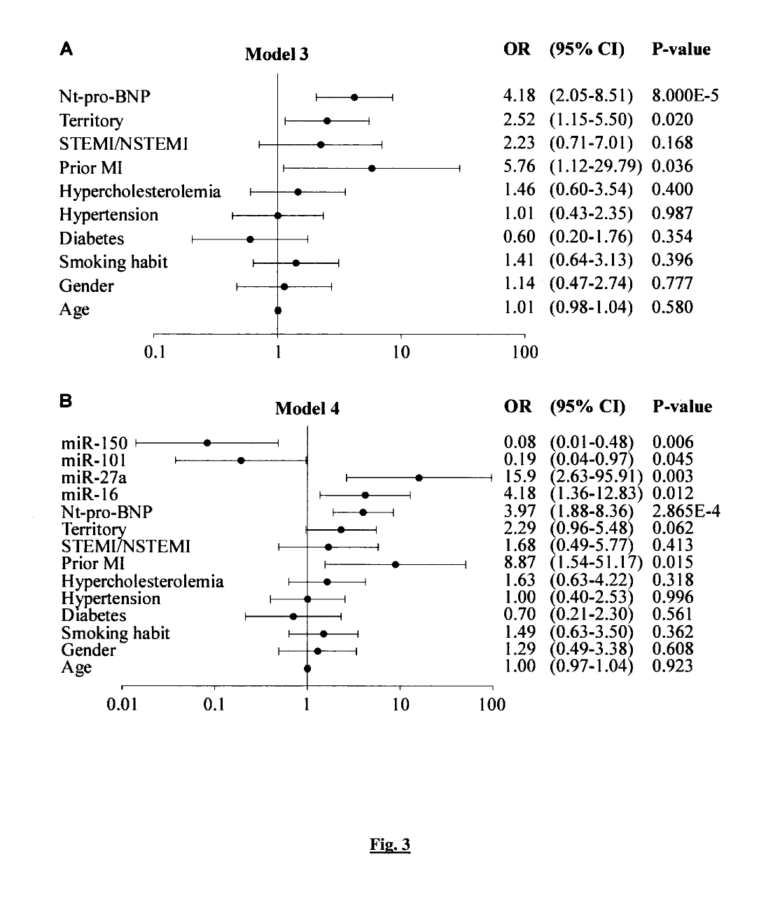

So far, WMIS was used as a continuous variable. To investigate whether miRNAs are valuable to predict whether a patient will or will not have impaired LV contractility, we dichotomized the population using a WMIS value of 1.2 as threshold, and we performed logistic regression analyses. These analyses were performed to model the probability (P) of belonging to the group of patients that will have impaired LV contractility (WMIS>1.2) over the probability of belonging to the group of patients that will not have impaired LV contractility (WMIS≦1.2). The logistic regression output can thereafter be used as a classifier by prescribing that a sample will be classified in the group of patient that will have impaired LV contractility if P is greater than 0.5, or 50%. Predicting variables included the expression levels of the four miRNAs (in log-scale), the level of Nt-pro-BNP and the other clinical variables. As for censored regression (model 1 and model 2), 2 multivariable models were built: model 3 includes all clinical variables and Nt-pro-BNP, and model 4 includes all variables of model 3 and the 4 miRNAs panel. Odds ratio (OR) for each variable in both models are shown in FIG. 3.

Probability (P) calculation of risk of impaired contractility from model 4 is done according to the following formulas:

P=exp(X)/(1+exp(X))

Whereas X=variable 1×ln OR (var 1)+variable 2×ln OR (var2)+ . . . variable X×ln OR (var x)+intercept

If P>0.5 then high risk of remodeling (WMIS>1.2)

If P<=0.5 then low or null risk of remodeling (WMIS<=1.2)

For miRNAs, “variable” in the formula indicates the expression values of this particular miRNA in the patient as determined by quantitative RT-PCR as described in Material and Methods.

For Nt-pro-BNP, “variable” in the formula indicates the concentration of Nt-pro-BNP in the patient blood as determined by immune-assay as described in Material and Methods.

For other clinical parameters, “variables” in the formula are binary, except for age which was considered as a continuous variable.

Odds ratio (OR) indicates the contribution of each variable to the prediction. OR below 1 indicates a negative association between a considered variable and the outcome of the patient; OR above 1 indicates a positive association between a considered variable and the outcome of the patient. OR were obtained with R version 2.13.1 with Hmisc, pROC, aod, lmtest and AER packages.

Each OR is associated with 95% CI and P value indicating the statistical significance of the variable in the model.

As an example, for model 4:

X=miR150×ln 0.08+miR101×ln 0.19+miR27a×ln 15.9+miR16×ln 4.18+Nt-pro-BNP×ln 3.97+territory×ln 2.29+STEM/NSTEMI×ln 1.68+Prior MI×ln 8.87+Hypercholesterolemia×ln 1.63+Hypertension×ln 1.00+Diabetes×ln 0.70+Smoking habit×ln 1.49+Gender×ln 1.29+Age×ln 1.00+ln 8.51×0E-5

Each value of the odd ratio as mentioned in the formula can be replaced by any value within its corresponding 95% CI. As an example from model 4, considering miR-150, OR value can be from 0.01 to 0.48. However, the intercept (ln 8.51×10E-5) is a constant.

Patients with anterior STEMI and elevated Nt-pro-BNP were at high risk of dyskinesia (FIG. 3A). All 4 miRNAs were significantly associated with WMIS group. Patients with low levels of miR-150/101 and elevated levels of miR-16/27a were at high risk of dyskinesia (FIG. 3B).

We next determined the added value of combinations of miRNAs. As shown in Table 4, the 4 miRNA panel had an additive value to the model with clinical parameters and Nt-pro-BNP (model 3). Adding the 4 miRNAs decreased the AIC from 188.269 to 181.432 (P=0.005). miR-27a/150 was the smallest combination of miRNAs which generated a significant added value.

TABLE 4
Added value of combinations of miRNAs (logistic regression)

| miRNA added to | Wald chi square | | LRT |
|---|---|---|---|
| model 3 | test P-value | AIC | P-value |
| None | 0.003 | 188.269 | |
| miR-16 | 0.003 | 188.381 | 0.169 |
| miR-27a | 0.003 | 186.591 | 0.055 |
| miR-101 | 0.004 | 189.476 | 0.373 |
| miR-150 | 0.004 | 190.261 | 0.931 |
| miR-16 + miR-27a | 0.005 | 188.245 | 0.134 |
| miR-16 + miR-101 | 0.006 | 190.332 | 0.380 |
| miR-16 + miR-150 | 0.003 | 187.753 | 0.105 |
| miR-27a + miR-101 | 0.005 | 186.842 | 0.066 |
| miR-27a + miR-150 | 0.004 | 186.117 | 0.046 |
| miR-101 + miR-150 | 0.006 | 191.080 | 0.552 |
| miR-16 + miR-27a + miR-101 | 0.007 | 187.837 | 0.092 |
| miR-16 + miR-27a + miR-150 | 0.003 | 183.838 | 0.015 |
| miR-16 + miR-101 + miR-150 | 0.005 | 189.380 | 0.180 |
| miR-27a + miR-101 + miR-150 | 0.006 | 186.389 | 0.049 |
| miR-16 + miR-27a + miR-101 + | 0.003 | 181.432 | 0.005 |
| miR-150 | | | |

Shown are the results of all combinations of miRNAs added to model 3. The Wald chi square test indicates the significance of the model. The likelihood ratio test (LRT) compares the predictive value of a model with miRNAs to model 1. AIC: Akaike information criteria.

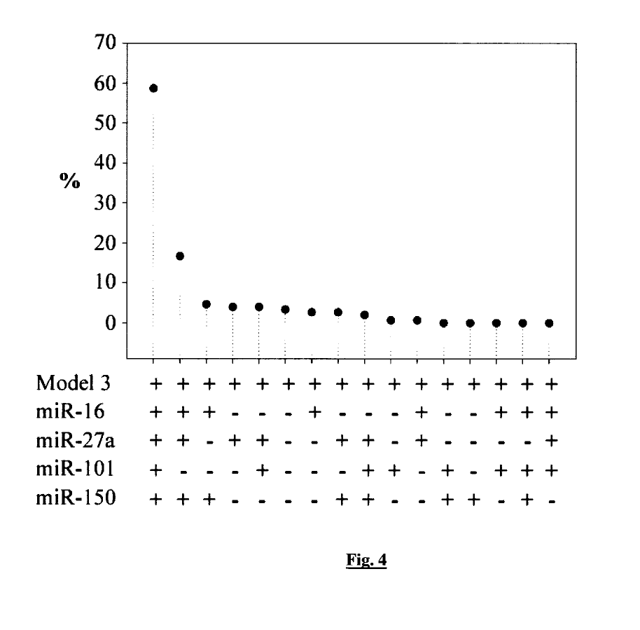

Bootstrap cross validation confirmed that the 4 miRNA panel provided the optimal improvement of prediction (FIG. 4).

### 6. Reclassification Analyses

The continuous version of the Net Reclassification Index and the Integrated Discrimination Improvement were computed to determine the ability of miRNAs to correctly reclassify patients misclassified by model 3 (Table 5). These are indexes of the change in classification of patients from one category of WMIS to another category (≦1.2 or >1.2). The 4 miRNA panel was able to reclassify a significant proportion of patients, as attested by a NRI of 66%(P=5×10E-5) and an IDI of 8% (P=0.001). Several combinations of miRNAs also provided significant reclassifications, such as miR-16/150, miR-27a/150, miR-16/27a1150, or miR-27a/101/150. However, no single miRNA had a significant reclassification capability.

TABLE 5
Reclassification analyses (logistic regression)

| | | | | | | IDI |
|---|---|---|---|---|---|---|
| miRNA added to | | | NRI | | | P- |
| model 3 | NRI | 95% CI | P-value | IDI | 95% CI | value |
| miR-16 | 0.179 | −0.142-0.499 | 0.275 | 0.010 | −0.007-0.027 | 0.243 |
| miR-27a | 0.263 | −0.057-0.584 | 0.108 | 0.017 | −0.007-0.040 | 0.162 |
| miR-101 | 0.181 | −0.139-0.502 | 0.267 | 0.004 | −0.007-0.015 | 0.453 |
| miR-150 | 0.120 | −0.201-0.440 | 0.464 | 1.53E−04 | −0.001-0.001 | 0.774 |
| miR-16 + miR-27a | 0.314 | −0.007-0.634 | 0.055 | 0.019 | −0.005-0.044 | 0.125 |
| miR-16 + miR-101 | 0.125 | −0.195-0.446 | 0.444 | 0.010 | −0.007-0.027 | 0.232 |
| miR-16 + miR-150 | 0.331 | 0.010-0.651 | 0.043 | 0.028 | 0.002-0.053 | 0.033 |
| miR-27a + miR-101 | 0.379 | 0.058-0.699 | 0.021 | 0.024 | −0.004-0.053 | 0.087 |
| miR-27a + miR-150 | 0.646 | 0.326-0.967 | 7.78E−05 | 0.031 | 0.001-0.061 | 0.046 |
| miR-101 + miR-150 | 0.213 | −0.108-0.533 | 0.194 | 0.007 | −0.006-0.021 | 0.296 |
| miR-16 + miR-27a + | 0.474 | 0.154-0.795 | 0.004 | 0.030 | −0.001-0.060 | 0.054 |
| miR-101 | | | | | | |
| miR-16 + miR-27a + | 0.415 | 0.095-0.736 | 0.011 | 0.056 | 0.018-0.095 | 0.004 |
| miR-150 | | | | | | |
| miR-16 + miR-101 + | 0.257 | −0.063-0.578 | 0.115 | 0.030 | 0.004-0.056 | 0.025 |
| miR-150 | | | | | | |
| miR-27a + miR- | 0.514 | 0.193-0.834 | 0.002 | 0.039 | 0.005-0.072 | 0.023 |
| 101 + miR-150 | | | | | | |
| miR-16 + miR-27a + | 0.663 | 0.342-0.983 | 5.05E−05 | 0.077 | 0.032-0.122 | 0.001 |
| miR-101 + miR-150 | | | | | | |

Shown are the results of all combinations of miRNAs added to model 3. The continuous version of the net reclassification index (NRI) was used in these analyses. CI: confidence interval. IDI: integrated discrimination improvement.

### 7. Classification of Patients with Ambiguous Phenotype

A main advantage of new biomarkers is to improve the classification of patients with intermediate phenotypes, which are difficult to classify using existing biomarkers. To test the accuracy of the 4 miRNA panel to improve the classification of patients with ambiguous phenotype, we considered borderline patients (l<WMIS<1.4, n=49). Among these, 25 patients had moderate LV dysfunction (1.2<WMIS<1.4) and 24 had no LV dysfunction (1<WMIS≦1.2). Logistic regression and leave-one-out cross validation were used in these analyses. Two models were built, one with clinical variables and Nt-pro-BNP and one with clinical variables, Nt-pro-BNP and the 4 miRNA panel. The model with clinical variables and Nt-pro-BNP had a specificity of 75%, but also a poor sensitivity of 48%. The 4 miRNAs panel increased the sensitivity to 60%, while maintaining the specificity at 75%. With miRNAs, the positive predictive value was increased from 67% to 71%, and the negative predictive value was increased from 58% to 64%. Therefore, the 4 miRNAs panel improved the prognostication of patients with ambiguous phenotype, particularly dyskinetic patients.

Materials and Methods

1. Patients

One hundred and fifty patients with ST-elevation AMI (STEMI) (Table 1) were enrolled in this study. The diagnosis of AMI was based on presentation with appropriate symptoms of myocardial ischemia, dynamic ST segment elevation, and increase in markers of myocyte necrosis [creatine kinase (CK) and troponin I (TnI)] to above twice the upper limit of the normal range. Venous blood samples were collected in EDTA-aprotinin tubes, immediately prior to discharge (day 3-4 after AMI). Samples were centrifuged within 30 minutes and plasma stored in aliquots at −80° C.

The protocol for both cohorts was approved by the local research ethics committee and written informed consent was obtained from all subjects. The conduct of the study was in accordance with the Declaration of Helsinki.

Patients were admitted to Glenfield Hospital, Leicester, UK between September 2004 and March 2005, and were enrolled in a prospective study of LV remodelling after AMI⁹. Half of these patients were treated by thrombolysis. None received primary percutaneous coronary intervention (PPCI), which was not in routine use at this centre at the time. Cardiac function was assessed by echocardiography, as described⁹, conducted by a single operator (DK) at discharge and at a median of 176 days (range 138-262 days) after AMI. LV contractility was evaluated by the LV wall motion index score (WMIS), using a standard 16-segment model from para-sternal long- and short-axis and apical two- and four-chamber views. Each LV segment was scored as 0, hyperkinetic; 1, normal; 2, hypokinetic; 3, akinetic; 4, dyskinetic. The total was divided by the number of segments analysed to give an overall score with higher values indicating more impaired LV contractility. In some analyses, patients were dichotomized into impaired LV contractility group (WMIS>1.2 at follow-up) and preserved LV contractility group (WMIS≦1.2 at follow-up).

2. Plasma miRNAs Determination

Total RNA was extracted from plasma samples using the mirVana PARIS kit (Ambion, Applied Biosystem, Lennik, Belgium) without enrichment for small RNAs. A mix of 3 spiked-in synthetic C. elegans miRNAs (Qiagen, Venlo, The Netherlands), lacking sequence homology to human miRNAs, was added to plasma samples for correction of extraction efficiency. Potential genomic DNA contamination was eliminated using DNase (Qiagen). Reverse transcription of RNA was performed with the miScript reverse transcription kit (Qiagen). The resulting cDNA was diluted 10-fold before quantitative PCR using the miScript SYBR-green PCR kit (Qiagen). miRNA-specific miScript primer sets were obtained from Qiagen. Expression values were normalized using the mean Ct obtained from the spiked-in controls [calculation formula: 2 exp (mean Ct spiked-in controls−Ct target miRNA)] and log-transformed. The detection limit of the PCR assay was −7.2, which is the log transformation of the minimum expression detected divided by 10.

3. Nt-Pro-BNP Assay

Peptides corresponding to the N-terminal (amino acids 1 to 12) and C-terminal (amino acids 65 to 76) of the human Nt-pro-BNP were used to raise rabbit polyclonal antibodies. IgG from the sera was purified on protein A sepharose columns. The C-terminal—directed antibody (0.5 μg in 100 μL for each well) was immobilized onto ELISA plates. The N-terminal antibody was affinity purified and biotinylated using biotin-X-N-hydroxysuccinimide ester (Calbiochem). Aliquots (20 μL) of samples or Nt-pro-BNP standards were incubated in the C-terminal antibody coated wells with the biotinylated antibody for 24 hours at 4° C. ELISA plates were washed with 0.1% Tween in PBS, and streptavidin (Chemicon International Ltd) labeled with methyl-acridinium ester (5×106 relative light units/mL) was added to each well. Plates were read on a Dynatech MLX Luminometer, with sequential injections of 100 μL of 0.1 mol/L nitric acid (with H2O2) and then 100 μL of NaOH (with cetyl ammonium bromide).13 The lower limit of detection was 14.4 fmol/mL of unextracted plasma. Within and between assays, coefficients of variation were acceptable at 2.3% and 4.8%, respectively. There was no cross-reactivity with ANP, BNP, or CNP.

4. Statistical Analysis

Patient Characteristics

Analysis of demographic features and echo parameters were carried out using SigmaPlot v 11.0. For all comparisons, a P-value<0.05 was considered significant. For categorical data, comparisons were by Chi-square test. Comparisons between groups of continuous data were performed with t-test for Gaussian data and the Mann-Whitney test on ranks for non-normally distributed data. Normality was assessed with the Shapiro-Wilk test.

Prediction Analyses

Prediction analyses were performed with R version 2.13.1 with Hmisc, pROC, aod, lmtest and AER packages. A P-value was considered significant when lower than 0.05. Clinical features were coded as 1 for presence and 0 for absence. Male was chosen as the reference level for sex in regression models. No data were missing thus no imputation method was performed.

Model Fitting

WMIS was first treated as a continuous variable (models 1 and 2). Since more than a third of the patients add a value that equalled one (the remaining patients having greater values), a left censored Tobit regression¹¹ was performed to model WMIS with different sets of predictors. WMIS was then dichotomized into two groups (WMIS<=1.2 and WMIS>1.2), which were analysed by logistic regression (models 3 and 4).

Model parameter estimates were tested for nullity using a Z test in censored regression and a Wald Chi-square test in logistic regression. Residuals were analysed graphically both to detect nonlinear relationships between each variable in a model and WMIS, and to check normality assumptions for Tobit regression. For logistic regression, odd ratios (OR) and 95% confidence intervals (CI) were obtained by exponential transformation, and are shown in FIGS. 1 to 3.

Best Model Selection

To determine which miRNA or combination of miRNAs had the maximal added value, all 15 possible combinations of miRNAs among the 4 miRNAs measured were generated and successively added to the reference model containing clinical parameters and Nt-pro-BNP. For each model, a Wald Chi-square test was used to assess the global effect of miRNAs on WMIS. The added value of miRNAs was tested for significance using the likelihood ratio test (LRT). In the dichotomous case, continuous net reclassification index (NRI) and integrated discrimination improvement (IDI)¹² were evaluated and tested for nullity. The final model was finally selected by minimizing Akaike Information Criterion (AIC) which is penalized by the number of variables added in the model to avoid overfitting.

Model Validation

Bootstrap internal validation was used to correct all measures of model performance for optimisation. For each bootstrap sample (i.e. a random sample of individuals with the same size as the original sample where a given patient can appear several times), the whole model selection process was performed again to select the best model according to AIC criterion; the original sample was then tested with this model. In order to evaluate optimisation, NRI and IDI were computed with the test (i.e. original) set and subtracted to the same measures computed with the bootstrap sample. Afterwards optimisation was averaged across 150 bootstrap replications and finally subtracted to the measures obtained with the original sample as a training set.

Borderline Patients Classification

Borderline patients were defined as having 1<WMIS<1.4. To determine whether miRNAs improved the classification of these patients, cross-validation was performed by successively leaving only those patients out one by one during logistic regression. A patient was classified as WMIS>1.2 when its probability was greater or equal to 0.5 and as WMIS<=1.2 otherwise. Sensitivity, specificity, positive and negative predictive values were then computed and compared exclusively for those patients between both models.

The present invention refers to the following nucleotide and amino acid sequences:

Nucleotide sequence encoding microRNA 16
(miRNA). (Accession Number of NCBI Reference
Sequence: NR_029486.1)
SEQ ID No. 1:
gtcagcagtg ccttagcagc acgtaaatat tggcgttaag
attctaaaat tatctccagt attaactgtg ctgctgaagt
aaggttgac
Nucleotide sequence encoding microRNA 27a
(miRNA). (Accession Number of NCBI Reference
Sequence: NR_029501.1)
SEQ ID No. 2:
ctgaggagca gggcttagct gcttgtgagc agggtccaca
ccaagtcgtg ttcacagtgg ctaagttccg ccccccag
Nucleotide sequence encoding microRNA 101
(miRNA). (Accession Number of NCBI Reference
Sequence: NR_029516.1)
SEQ ID No. 3:
tgccctggct cagttatcac agtgctgatg ctgtctattc
taaaggtaca gtactgtgat aactgaagga tggca
Nucleotide sequence encoding microRNA 150
(miRNA). (Accession Number of NCBI
Reference Sequence: NR_029703.1)
ctccccatgg ccctgtctcc caacccttgt accagtgctg
ggctcagacc ctggtacagg cctgggggac agggacctgg
ggac
Amino acid sequence encoding Nt-pro-BNP
(Accession Number of NCBI Reference Sequence:
NP_002512.1)
SEQ ID No. 5:
mdpqtapsra lllllflhla flggrshplg spgsasdlet
sglqeqrnhl qgklselqve qtsleplqes prptgvwksr
evategirgh rkmvlytlra prsplunvqgs gcfgrkmdri
ssssglgckv lrrh

### REFERENCES

1. Torabi A, Cleland J G, Khan N K, Loh P H, Clark A L, Alamgir F, Caplin J L, Rigby A S, Goode K. The timing of development and subsequent clinical course of heart failure after a myocardial infarction. Eur Heart J 2008; 29:859-870.
2. Talwar S, Squire I B, Downie P F, McCullough A M, Campton M C, Davies J E, Barnett D B, Ng L L. Profile of plasma N-terminal proBNP following acute myocardial infarction; correlation with left ventricular systolic dysfunction. Eur Heart J 2000; 21:1514-1521.
3. Gilad S, Meiri E, Yogev Y, Benjamin S, Lebanony D, Yerushalmi N, Benjamin H, Kushnir M, Cholakh H, Melamed N, Bentwich Z, Hod M, Goren Y, Chajut A. Serum microRNAs are promising novel biomarkers. PLoS One 2008; 3:e3148.
4. Mitchell P S, Parkin R K, Kroh E M, Fritz B R, Wyman S K, Pogosova-Agadjanyan E L, Peterson A, Noteboom J, O'Briant K C, Allen A, Lin D W, Urban N, Drescher C W, Knudsen B S, Stirewalt D L, Gentleman R, Vessella R L, Nelson P S, Martin D B, Tewari M. Circulating microRNAs as stable blood-based markers for cancer detection. Proc Natl Acad Sci USA 2008; 105:10513-10518.
5. Creemers E E, Tijsen A J, Pinto Y M. Circulating MicroRNAs: Novel Biomarkers and Extracellular Communicators in Cardiovascular Disease? Circ Res 2012; 110:483-495.
6. Devaux Y, Vausort M, Goretti E, Nazarov P V, Azuaje F, Gilson G, Corsten M F, Schroen B, Lair M L, Heymans S, Wagner D R. Use of Circulating MicroRNAs to Diagnose Acute Myocardial Infarction. Clin Chem 2012; 58:559-567.
7. Widera C, Gupta S K, Lorenzen J M, Bang C, Bauersachs J, Bethmann K, Kempf T, Wollert K C, Thum T. Diagnostic and prognostic impact of six circulating microRNAs in acute coronary syndrome. J Mol Cell Cardiol 2011; 51:872-875.
8. Zile M R, Mehurg S M, Arroyo J E, Stroud R E, Desantis S M, Spinale F G. Relationship Between The Temporal Profile of Plasma microRNA and Left Ventricular Remodeling In Patients Following Myocardial Infarction. Circ Cardiovasc Genet 2011; 4:614-619.
9. Kelly D, Cockerill G, Ng L L, Thompson M, Khan S, Samani N J, Squire I B. Plasma matrix metalloproteinase-9 and left ventricular remodelling after acute myocardial infarction in man: a prospective cohort study. Eur Heart J 2007; 28:711-718.
10. Omland T, Persson A, Ng L, O'Brien R, Karlsson T, Herlitz J, Hartford M, Caidahl K. N-terminal pro-B-type natriuretic peptide and long-term mortality in acute coronary syndromes. Circulation 2002; 106:2913-2918.
11. Tobin J. Estimation of relationships for limited dependent variables. Econometrica 1958; 26:24-36.
12. Pencina M J, D'Agostino R B, Sr., D'Agostino R B, Jr., Vasan R S. Evaluating the added predictive ability of a new marker: from area under the ROC curve to reclassification and beyond. Stat Med 2008; 27:157-172; discussion 207-112.
13. Zampetaki A, Willeit P, Tilling L, Drozdov I, Prokopi M, Renard J M, Mayr A, Weger S, Schett G, Shah A, Boulanger C M, Willeit J, Chowienczyk P T, Kiechl S, Mayr M. Prospective Study on Circulating MicroRNAs and Risk of Myocardial Infarction. J Am Coll Cardiol 2012; 60:290-299.
14. Zhu H, Fan G C. Role of microRNAs in the reperfused myocardium towards post-infarct remodelling. Cardiovasc Res 2012; 94:284-292.
15. Abdellatif M. Differential Expression of MicroRNAs in Different Disease States. Circ Res 2012; 110:638-650.
16. Da Costa Martins P A, De Windt L J. MicroRNAs in control of cardiac hypertrophy. Cardiovasc Res 2012; 93:563-572.
17. Yamamoto K, Ohki R, Lee R T, Ikeda U, Shimada K. Peroxisome Proliferator-Activated Receptor γ Activators Inhibit Cardiac Hypertrophy in Cardiac Myocytes. Circulation 2001; 104:1670-1675.
18. Belevych A E, Sansom S E, Terentyeva R, Ho H-T, Nishijima Y, Martin M M, Jindal H K, Rochira J A, Kunitomo Y, Abdellatif M, Carnes C A, Elton T S, Gyorke S, Terentyev D. MicroRNA-1 and -133 Increase Arrhythmogenesis in Heart Failure by Dissociating Phosphatase Activity from RyR2 Complex. PLoS ONE 2011; 6:e28324.
19. Yang B, Lin H, Xiao J, Lu Y, Luo X, Li B, Zhang Y, Xu C, Bai Y, Wang H, Chen G, Wang Z. The muscle-specific microRNA miR-1 regulates cardiac arrhythmogenic potential by targeting GJA1 and KCNJ2. Nat Med 2007; 13:486-491.
20. Aurora A B, Mahmoud A I, Luo X, Johnson B A, van Rooij E, Matsuzaki S, Humphries K M, Hill J A, Bassel-Duby R, Sadek H A, Olson E N. MicroRNA-214 protects the mouse heart from ischemic injury by controlling Ca2+ overload and cell death. The Journal of Clinical Investigation 2012; 122:1222-1232.
21. Nishi H, Ono K, Hone T, Nagao K, Kinoshita M, Kuwabara Y, Watanabe S, Takaya T, Tamaki Y, Takanabe-Mori R, Wada H, Hasegawa K, Iwanaga Y, Kawamura T, Kita T, Kimura T. MicroRNA-27a Regulates Beta Cardiac Myosin Heavy Chain Gene Expression by Targeting Thyroid Hormone Receptor β1 in Neonatal Rat Ventricular Myocytes. Molecular and Cellular Biology 2011; 31:744-755.
22. Sadegh M K, Ekman M, Rippe C, Uvelius B, Sward K, Albinsson S. Deletion of Dicer in Smooth Muscle Affects Voiding Pattern and Reduces Detrusor Contractility and Neuroeffector Transmission. PLoS ONE 2012; 7:e35882.
23. Pan Z, Sun X, Shan H, Wang N, Wang J, Ren J, Feng S, Xie L, Lu C, Yuan Y, Zhang Y, Wang Y, Lu Y, Yang B. miR-101 Inhibited Post-Infarct Cardiac Fibrosis and Improved Left Ventricular Compliance via FOS/TGFβ1 Pathway. Circulation 2012.
24. Tijsen A J, Pinto Y M, Creemers E E. Non-cardiomyocyte microRNAs in heart failure. Cardiovasc Res 2012; 93:573-582.
25. Senn S, Julious S. Measurement in clinical trials: a neglected issue for statisticians? Stat Med 2009; 28:3189-3209.
26. Fedorov V, Mannino F, Zhang R. Consequences of dichotomization. Pharm Stat 2009; 8:50-61.

### Example 2

### Identification of miRNAs of the Invention

The selection of the 4 miRNAs of the present invention was a long process, starting from an initial hypothesis that circulating miRNAs may be associated with remodelling post MI. The first step was to perform microarray experiments in blood samples from 2 small groups of MI patients, one with, and one without, remodelling. From the 695 miRNAs represented on the microarrays, we isolated 271 miRNAs that were differentially expressed between patients with and without remodelling. The complete data are in Table S1. To isolate from these 271 those with potential link to remodelling, we used a systems-based approach with interaction networks. This permitted the identification of 10 miRNAs with the highest probability of association with remodelling. From these, we selected miR-27a/-101/-150 because of their high level of expression and differential expression between remodelers and non-remodelers. Also included, and that were not derived from the systems-based approach, were miR-16/-92a/486, because of their high expression and differential expression in microarrays, and because they had been noted in Goretti et al., J. Leukoc. Biol. (2013). We then measured these 6 miRNAs in 150 MI patients and observed that the 4 miRNAs of the invention, miR-16/27a/101/150 provide a predictive value over and above BNP.

In more detail:

Procedure Used to Select the miRNAs of the Invention

The working hypothesis was that circulating miRNAs are useful to predict left ventricular remodelling and clinical outcome after myocardial infarction (MI).

Multi-Step Procedure.

Microarray experiments ((Devaux, Y., Vausort, M, McCann, G. P., Zangrando, J., Kelly, D., Razvi, N., Zhang, L., Ng, L. L., Wagner, D. R, Squire, I. B. (2013) MicroRNA-150. A novel marker of left ventricular remodeling after acute myocardial infarction. Circ. Cardiovasc Genet. 6:290-298) Circulating miRNA expression profiles were established by using blood samples obtained at hospital discharge from 2 groups of 30 MI patients enrolled at the Leicester Hospital in UK (=derivation cohort). Patients were classified depending on whether they showed left ventricular (LV) remodelling at 6-month follow-up. LV function was assessed by echocardiographic analysis, and LV remodelling was assessed by the change (ΔEDV) in left ventricular end diastolic volume (LVEDV) between discharge and follow-up. LV remodelling was defined as any increase in LVEDV during follow-up. Patients with diabetes or a prior MI event were excluded. Patients with or without LV remodelling were matched by age, and had comparable cardiovascular risk factors. The rationale for this selection procedure was to avoid any bias due to a potential effect of confounding factors on circulating levels of miRNAs. Venous blood was collected in tubes citrated prior to hospital discharge [median of 176 (range 138-262) days after MI]. Plasma was harvested by centrifugation and stored at −80° C. until assayed. Identical volumes of plasma samples from each of the 30 patients of a specific group were pooled to reach a final volume of 400 μL for each group. This pooling strategy has been described elsewhere¹. The two pools were processed conjointly. Total RNA was extracted from these 2 pools of plasma using miRVana PARIS isolation kit (Applied Biosystems, Lennik, Belgium), dephosphorylated and labelled using miRNA Complete Labelling and hybridisation kit (Agilent Technologies, Massy, France). Hybridisation was performed on microarrays covering 695 miRNAs (Human Microarray Release 12.0 slides from Agilent Technologies, Santa Clara, Calif.). Four arrays were hybridised per group. Scanning was achieved with the Genepix 4000B Scanner (Molecular Devices, Sunnyvale, USA). Raw data were acquired with the Genepix Pro software (Molecular Devices). Spots flagged as absent and having a signal to noise ratio less than 3 were removed. Median values of replicate probes were background subtracted. Normalisation was performed by using normalizeQuantile function from the Bioconductor limma package². The miRNAs detected on at least 2 arrays of each group were selected for further analysis. Microarray data are presented in Table S1.

Of the 695 miRNAs represented on microarrays, 160 miRNAs were detected in 1 or both groups of patients. Twenty-nine miRNAs were detected only in patients without remodelling. Overall, 271 miRNAs were differentially expressed between patients with and without remodelling (false discovery rate<0.05 using Statistical Analysis of Microarrays software; See Table S1). To isolate among these 271 miRNAs those with the highest probability of being associated with remodelling post MI, we used a systems-based approach.

Systems-based approach (Devaux, Y., Vausort, M, McCann, G. P., Zangrando, J., Kelly, D., Razvi, N., Zhang, L., Ng, L. L., Wagner, D. R:, Squire, I. B. (2013) MicroRNA-150. A novel marker of left ventricular remodeling after acute myocardial infarction. Circ. Cardiovasc Genet. 6:290-298)

1. We searched for seed genes known to be related to remodelling from NCBI gene database using the keywords: “ventricular remodelling” and “myocardial infarction”. The search was limited to human genes. Proteins known to interact with the proteins encoded by seed genes were identified from the IntAct⁷, DIP⁸ and MINT⁹ databases. Only the interactions found in at least two databases were selected for further analysis. Then all seed and interacting proteins were used as inputs to TargetScan³, PicTar⁴ and MicroCosm⁵ databases to retrieve miRNAs predicted to have binding sites in the genes encoding these proteins. Gene annotation and identification of enriched KEGG (Kyoto Encyclopedia of Genes and Genomes) pathways were performed using the Database for Annotation, Visualisation and Integrated Discovery (DAVID)⁶. Only the miRNA-target pairs found in at least two databases were selected to build interaction networks. Networks were visualised with CytoScape¹⁰ and Polar Mapper¹¹. Node traffic estimation was computed with Polar Mapper. High traffic nodes can represent “cross-communication” hotspots or “bottlenecks” in the network.

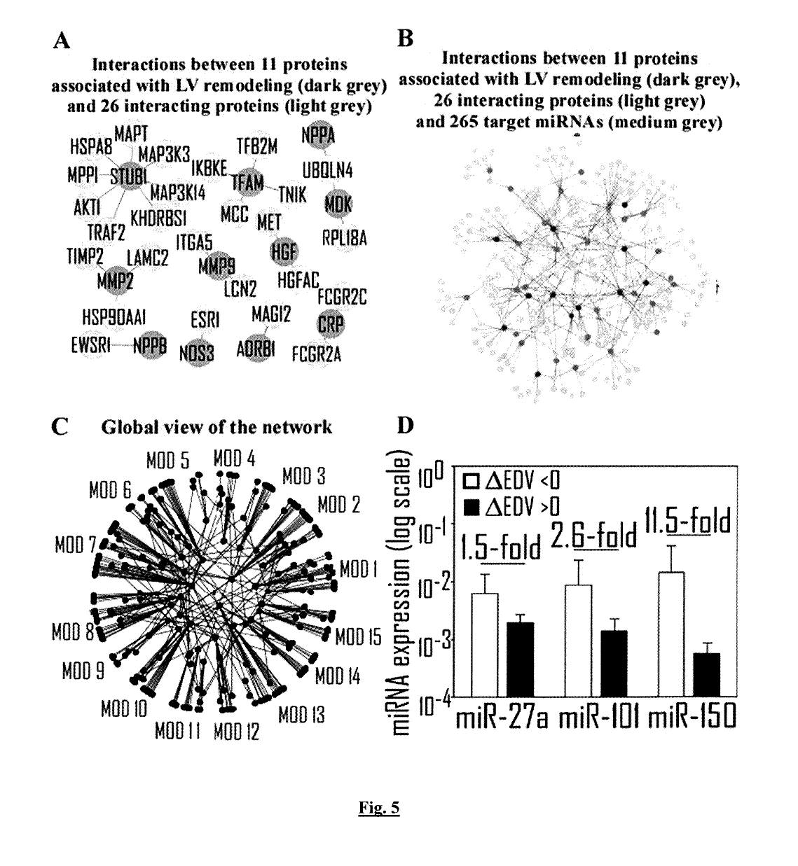

Then we used these 13 seed proteins to retrieve from protein-protein interaction databases a list of 26 proteins (=interactors) known to interact with the 13 remodelling genes. See Table 6 and FIG. 5A.

Then, we retrieved from miRNA-target databases a list of 265 miRNAs (Table 7) predicted to target the 13 seed genes or the genes encoding the 26 interacting proteins (Table 6). Using these genes and miRNAs, we built an interaction network (FIGS. 5B and 5C) in which nodes represent either proteins or miRNAs and edges represent either protein-protein interactions or miRNA-target pairs. Bioinformatic analysis of this network allowed isolation of the top 10 high-traffic miRNAs (hsa-miR-27a/-133a/-625/-296-3p/-31/-23a/-204/-19a/-101/-150). These miRNAs were predicted to regulate the expression of a high number of genes encoding proteins involved in remodelling and therefore potentially interesting as predictors of outcome after MI.

From these 10 high-traffic miRNAs, we selected miR-27a/-101/-150 because of their high level of expression and differential expression between remodelers and non-remodelers in the microarray experiments from 1. Differential expression between remodelers and non-remodelers was confirmed by quantitative PCR (FIG. 5D).

FIG. 5 shows systems-based identification of candidate miRNAs. FIG. 5 A. Network of interactions between proteins known to be associated with LV remodelling in humans (dark grey nodes) and 26 interacting proteins (light grey). From the 13 proteins associated with LV remodelling, only 11 had known protein-protein interactions in at least 2 queried databases. B. Network of interactions between the 11 proteins associated with LV remodelling (dark grey), their 26 interactors light grey) and their 265 target miRNAs (medium grey). This network was built with CytoScape. C. Global view of the network containing 15 modules. This view was built with Polar Mapper. D. Discharge plasma levels of miR-27a/101/150 in 60 AMI patients of the derivation cohort, as measured by quantitative PCR. Patients with decreased end-diastolic volume between discharge and follow-up (ΔEDV<0, n=30) had higher levels of miRNAs compared to patients with increased EDV (ΔEDV>0, n=30). Means±95% CI are shown.

2. Selection of miR-16/-92a/486

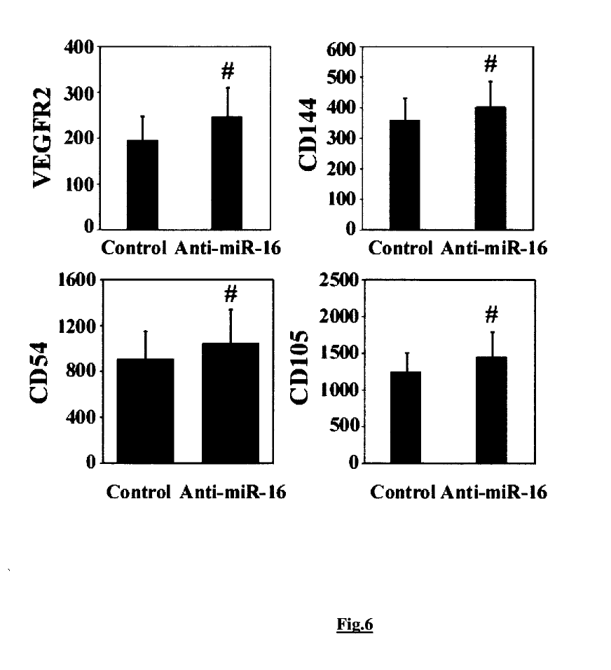

We also selected miR-16/-92a/486, which were not derived from the systems-based approach, for the following reasons:
high level of expression in microarrays from 1 (Table S1)
strong differential expression between remodelers and non-remodelers in microarrays from 1 (Table S1)
we previously showed that miR-16 played a role in the differentiation of endothelial progenitor cells, thus having a potential effect on remodelling post MI (FIG. 6 and Goretti et al J Leukoc Biol 2013 May; 93(5):645-55).

FIG. 6 shows expression of differentiation-related genes in early endothelial progenitor cells treated by anti-miR-16. Early endothelial progenitor cells obtained 4 days after plating PBMCs onto human fibronectin-coated plates were transfected with 30 nmol/1 anti-miR-16, and expression of endothelial markers was assessed by flow cytometry after 24 h. Results are mean±SD (n=3). #P<0.05. Comparisons are between control and anti-miR-16 conditions.

3. Assessment of miR-16/-27a/-92a/-101/-150/486

We measured the plasma levels of these 6 miRNAs at hospital discharge in 150 MI patients from the Leicester Hospital and we evaluated their ability to predict remodelling, as assessed by the wall motion index score (index of cardiac contractility) obtained by echocardiography at 6-month follow-up. Nt-pro-BNP, a known predictor of outcome post MI, was also measured. Using multiple logistic regression, we deduced that LV contractility (dependent variable 1=WMIS<=1,2) can be predicted from a linear combination of the 4 miRNAs miR-16/27a/101/150 and Nt-pro-BNP as demonstrated below.

Backward Stepwise

Regression:

Data Source:

Data 1 in Log reg WMIS miRs BNP 150 UK echo

Dependent Variable: 1 = WMIS <= 1.2
F-to-Enter: 4.000 P = 0.047
F-to-Remove: 3.900 P = 0.050

Step 0:
Standard Error of Estimate = 0,442
Analysis of Variance:

| Group | DF | SS | MS | F | P |
|---|---|---|---|---|---|
| Regression | 7 | 9.897 | 1.414 | 7.247 | <0.001 |
| Residual | 148 | 28.872 | 0.195 | | |

Variables in Model

| | | Std. | Std. | F-to- | |
|---|---|---|---|---|---|
| Group | Coef. | Coeff. | Error | Remove | P |
| Constant | 1.704 | | 0.408 | | |
| Log NTproBNP | −0.216 | −0.312 | 0.0524 | 16.996 | <0.001 |
| dis | | | | | |
| miR-92a | 0.119 | 0.115 | 0.318 | 0.140 | 0.708 |
| miR-16 | −0.227 | −0.271 | 0.167 | 1.844 | 0.176 |
| miR-27a | −0.598 | −0.559 | 0.168 | 12.748 | <0.001 |
| miR-150 | 0.506 | 0.424 | 0.148 | 11.620 | <0.001 |
| miR-486 | −0.236 | −0.228 | 0.256 | 0.848 | 0.359 |
| miR-101 | 0.345 | 0.349 | 0.142 | 5.908 | 0.016 |

Variables not in Model

| | | |
|---|---|---|
| Group | F-to-Enter | P |

Step 1: miR-92a Removed

| | | |
|---|---|---|
| R = 0.505 | Rsqr = 0.255 | Adj Rsqr = 0.225 |
| Standard Error of Estimate = 0.440 | | |

Analysis of Variance:

| Group | DF | SS | MS | F | P |
|---|---|---|---|---|---|
| Regression | 6 | 9.870 | 1.645 | 8.481 | <0.001 |
| Residual | 149 | 28.900 | 0.194 | | |

Variables in Model

| | | Std. | Std. | F-to- | |
|---|---|---|---|---|---|
| Group | Coef. | Coeff. | Error | Remove | P |
| Constant | 1.687 | | 0.404 | | |
| Log NTproBNP | −0.213 | −0.308 | 0.0517 | 17.007 | <0.001 |
| dis | | | | | |
| miR-16 | −0.194 | −0.232 | 0.142 | 1.873 | 0.173 |
| miR-27a | −0.574 | −0.537 | 0.155 | 13.776 | <0.001 |
| miR-150 | 0.509 | 0.427 | 0.148 | 11.894 | <0.001 |
| miR-486 | −0.166 | −0.160 | 0.174 | 0.905 | 0.343 |
| miR-101 | 0.336 | 0.340 | 0.140 | 5.802 | 0.017 |

Variables not in Model

| Group | F-to-Enter | P |
|---|---|---|
| miR-92a | 0.140 | 0.708 |

Step 2: miR-486 Removed

| | | |
|---|---|---|
| R = 0.500 | Rsqr = 0.250 | Adj Rsqr = 0.225 |
| Standard Error of Estimate = 0.440 | | |

Analysis of Variance:

| Group | DF | SS | MS | F | P |
|---|---|---|---|---|---|
| Regression | 5 | 9.694 | 1.939 | 10.002 | <0.001 |
| Residual | 150 | 29.075 | 0.194 | | |

Variables in Model

| | | Std. | Std. | F-to- | |
|---|---|---|---|---|---|
| Group | Coef. | Coeff. | Error | Remove | P |
| Constant | 1.824 | | 0.377 | | |
| Log NTproBNP | −0.222 | −0.321 | 0.0509 | 19.104 | <0.001 |
| dis | | | | | |
| miR-16 | −0.295 | −0.354 | 0.0933 | 10.024 | 0.002 |
| miR-27a | −0.526 | −0.492 | 0.146 | 12.955 | <0.001 |
| miR-150 | 0.474 | 0.398 | 0.143 | 11.006 | 0.001 |
| miR-101 | 0.297 | 0.300 | 0.133 | 4.966 | 0.027 |

Variables not in Model

| Group | F-to-Enter | P |
|---|---|---|
| miR-92a | 0.192 | 0.662 |
| miR-486 | 0.905 | 0.343 |

Summary Table

| Step # | Vars. | | | | | |
|---|---|---|---|---|---|---|
| Model | Entered | Vars. Removed | R | RSqr | Delta RSqr | Vars in |
| 1 | | miR-92a | 0.505 | 0.255 | 0.255 | 6 |
| 2 | | miR-486 | 0.500 | 0.250 | −0.0453 | 5 |

The dependent variable 1=WMIS<=1,2 can be predicted from a linear combination of the independent variables:

| | P |
|---|---|
| Log NTproBNP | <0.001 |
| dis | |
| miR-16 | 0.002 |
| miR-27a | <0.001 |
| miR-150 | 0.001 |
| miR-101 | 0.027 |

The following variables did not significantly add to the ability of the equation to predict 1=WMIS<=1,2 and were not included in the final equation: miR-92a miR-486

Normality Test (Shapiro-Wilk) Failed (P = <0.001)
Constant Variance Test: Passed (P = 0.352)
Power of performed test with alpha = 0.050:1.000

In the following Table S1, the abbreviation RMDG is used for ‘remodelling’.

TABLE S1

| | | | | | | | | | | Fold |
|---|---|---|---|---|---|---|---|---|---|---|
| | | No | No | No | No | | | | | change RMDG/ |
| Probe ID | miRNA name | RMDG 1 | RMDG 2 | RMDG 3 | RMDG 4 | RMDG 1 | RMDG 2 | RMDG 3 | RMDG 4 | no RMDG |
| A_25_P00010086 | hsa-let-7a | 2348.98 | 1813.55 | 1951.14 | 1864.58 | 1483.14 | — | — | — | |
| A_25_P00011584 | hsa-let-7a | 8741.11 | 5195.03 | 4958.22 | 4485.24 | 1797.61 | 1776.44 | 1665.98 | 1362.37 | 0.28 |
| A_25_P00010070 | hsa-let-7b | 29444.14 | 22846.62 | 23211.94 | 25074.20 | 4104.81 | 2747.05 | 3027.13 | 2761.04 | 0.13 |
| A_25_P00010071 | hsa-let-7b | 21619.43 | 14962.94 | 15872.33 | 15779.70 | 2173.01 | 1363.65 | 1846.21 | 1552.00 | 0.10 |
| A_25_P00010072 | hsa-let-7c | 6851.21 | 3505.88 | 5691.86 | 5314.67 | — | 1217.73 | — | — | |
| A_25_P00010073 | hsa-let-7c | 1466.53 | — | 1436.06 | — | — | — | — | 790.67 | |
| A_25_P00011981 | hsa-let-7d | 4149.71 | 2870.29 | 2733.51 | 2595.09 | — | — | — | — | |
| A_25_P00013126 | hsa-let-7d* | 1794.51 | 1636.57 | 1587.80 | 1439.40 | — | — | — | — | |
| A_25_P00013127 | hsa-let-7d* | 3714.96 | 3209.46 | 2753.13 | 2796.02 | 1990.35 | — | 1625.83 | 1776.97 | 0.57 |
| A_25_P00010088 | hsa-let-7f | 6214.62 | 4253.34 | 3421.54 | 3243.53 | 2063.05 | 1560.59 | — | 1713.79 | 0.44 |
| A_25_P00010089 | hsa-let-7f | 1391.82 | 970.73 | 1429.16 | 923.72 | — | — | — | — | |
| A_25_P00012141 | hsa-let-7g | 4936.85 | 3405.47 | 2446.58 | 2247.11 | — | — | — | — | |
| A_25_P00012142 | hsa-let-7g | 24703.11 | 19365.82 | 13713.56 | 13196.24 | 2343.21 | 1793.69 | 1868.19 | 1483.62 | 0.11 |
| A_25_P00012145 | hsa-let-7i | 15147.29 | 11074.96 | 9018.23 | 9361.91 | 2035.84 | 1624.48 | 1888.55 | 1843.63 | 0.17 |
| A_25_P00012146 | hsa-let-7i | 28531.77 | 24780.89 | 18496.01 | 19146.15 | 4466.45 | 3135.30 | 3718.18 | 3643.07 | 0.16 |
| A_25_P00012038 | hsa-miR-101 | 2735.74 | 1997.93 | 1712.68 | 1670.16 | — | — | — | — | |
| A_25_P00012039 | hsa-miR-101 | 4982.77 | 3882.79 | 2885.33 | 2925.49 | — | — | — | — | |
| A_25_P00011004 | hsa-miR-103 | 3862.08 | 3135.30 | 2188.26 | 2197.61 | — | — | — | — | |
| A_25_P00011005 | hsa-miR-103 | 1826.47 | 1802.20 | 1367.90 | 1400.14 | — | — | — | — | |
| A_25_P00010433 | hsa-miR-106b | 11412.79 | 9951.53 | 7114.67 | 6760.01 | 1076.79 | 2799.07 | 1592.19 | — | 0.21 |
| A_25_P00010434 | hsa-miR-106b | 4320.39 | 3180.05 | 2869.00 | 2615.21 | — | — | — | — | |
| A_25_P00011068 | hsa-miR-107 | 7748.12 | 5516.95 | 5584.48 | 4967.67 | 1734.63 | — | 1920.62 | 1795.85 | 0.30 |
| A_25_P00011069 | hsa-miR-107 | 25772.17 | 21370.36 | 16326.74 | 16550.84 | 6495.38 | 5157.09 | 5323.53 | 5481.51 | 0.28 |
| A_25_P00015059 | hsa-miR-1181 | 2614.28 | 2441.73 | 1738.60 | 1826.04 | — | — | — | — | |
| A_25_P00015060 | hsa-miR-1181 | 1729.27 | 1523.81 | 1402.67 | 1373.74 | — | — | — | — | |
| A_25_P00015061 | hsa-miR-1182 | 2719.90 | 2230.77 | 1803.18 | 1850.66 | — | — | — | — | |
| A_25_P00015062 | hsa-miR-1182 | 2434.84 | 2006.95 | 1862.48 | 1777.42 | — | — | — | — | |
| A_25_P00015063 | hsa-miR-1183 | 4540.49 | 4390.77 | 3767.45 | 3552.81 | — | — | — | — | |
| A_25_P00015064 | hsa-miR-1183 | 4189.01 | 4020.32 | 3887.94 | 3764.22 | — | — | 1230.46 | — | |
| A_25_P00015075 | hsa-miR-1202 | 3240.56 | 2418.10 | 3025.69 | 2866.50 | 938.71 | 1170.07 | 1246.36 | 1109.92 | 0.39 |
| A_25_P00015076 | hsa-miR-1202 | 3104.27 | 2506.86 | 2866.50 | 3025.69 | 825.38 | 919.91 | 1123.54 | 1053.13 | 0.34 |
| A_25_P00015087 | hsa-miR- | 3487.71 | 2720.90 | 3475.83 | 3475.83 | 1092.89 | 1618.86 | 1363.92 | 1217.66 | 0.40 |
| | 1207-5p | | | | | | | | | |
| A_25_P00015088 | hsa-miR- | 2982.72 | 2301.64 | 2697.50 | 3190.24 | 1039.75 | 1407.90 | 961.59 | 955.03 | 0.39 |
| | 1207-5p | | | | | | | | | |
| A_25_P00012153 | hsa-miR-122 | 1593.40 | 1292.34 | 1227.08 | 1216.36 | 2621.78 | 2293.57 | 2290.89 | 2246.78 | 1.77 |
| A_25_P00012154 | hsa-miR-122 | 2808.00 | 2364.15 | 2135.90 | 1984.81 | 4607.61 | 3351.48 | 4166.29 | 4043.84 | 1.74 |
| A_25_P00014906 | hsa-miR- | 3523.91 | 3089.34 | 2697.20 | 2523.95 | — | — | — | — | |
| | 1224-5p | | | | | | | | | |
| A_25_P00014907 | hsa-miR- | 3066.92 | 2587.07 | 2297.90 | 2310.16 | — | 1167.47 | — | — | |
| | 1224-5p | | | | | | | | | |
| A_25_P00014920 | hsa-miR- | 47509.43 | 38955.59 | 39596.53 | 35820.80 | 25235.45 | 32707.22 | 23978.31 | 23709.73 | 0.65 |
| | 1225-5p | | | | | | | | | |
| A_25_P00014921 | hsa-miR- | 36379.03 | 35625.04 | 33608.27 | 34647.35 | 21680.51 | 29327.63 | 22019.22 | 20110.79 | 0.66 |
| | 1225-5p | | | | | | | | | |
| A_25_P00015003 | hsa-miR- | 2528.64 | 2121.39 | 1771.38 | 1610.52 | — | — | — | — | |
| | 1226* | | | | | | | | | |
| A_25_P00015004 | hsa-miR- | 2228.36 | 2067.72 | 1339.53 | 1386.57 | — | — | — | — | |
| | 1226* | | | | | | | | | |
| A_25_P00014936 | hsa-miR-1228 | 1535.41 | 1316.02 | 1989.08 | 1954.01 | 1750.32 | — | — | 1371.17 | 0.91 |
| A_25_P00014937 | hsa-miR-1228 | 1203.63 | — | 1625.02 | 1511.94 | — | — | — | — | |
| A_25_P00014952 | hsa-miR-1234 | 1298.36 | — | — | 1168.64 | — | — | — | — | |
| A_25_P00014953 | hsa-miR-1234 | — | — | 1237.89 | 1413.51 | — | — | — | 1147.71 | |
| A_25_P00015142 | hsa-miR-1246 | 4372.21 | 2780.17 | 2061.17 | 2084.74 | — | — | — | — | |
| A_25_P00015143 | hsa-miR-1246 | — | — | 49827.70 | 49732.79 | 13789.26 | 14503.64 | 13879.65 | 11642.95 | 0.30 |
| A_25_P00015148 | hsa-miR-1249 | 1954.22 | 1711.48 | 2380.33 | 2286.38 | — | — | 1436.34 | 1413.07 | 0.72 |
| A_25_P00015149 | hsa-miR-1249 | 1720.73 | 1722.63 | 2214.96 | 2143.35 | — | — | — | — | |
| A_25_P00015158 | hsa-miR-1254 | 1106.16 | — | — | 1010.82 | — | — | — | — | |
| A_25_P00012212 | hsa-miR- | 1503.71 | 1644.45 | 1592.97 | 1724.45 | — | — | — | — | |
| | 125a-3p | | | | | | | | | |
| A_25_P00012213 | hsa-miR- | 1785.97 | 1652.03 | 1564.09 | 1592.06 | — | 181.00 | — | — | |
| | 125a-3p | | | | | | | | | |
| A_25_P00013941 | hsa-miR- | 1435.95 | 1574.28 | 1647.87 | 1547.01 | — | — | — | — | |
| | 125a-3p | | | | | | | | | |
| A_25_P00012215 | hsa-miR-126 | 2876.51 | 2404.97 | 2008.88 | 2002.34 | 1420.94 | — | — | — | |
| A_25_P00012216 | hsa-miR-126 | 6308.96 | 5133.04 | 3817.96 | 3815.63 | 2731.73 | 1544.16 | 2340.10 | 1915.93 | 0.45 |
| A_25_P00010600 | hsa-miR-126* | 1802.16 | 1409.89 | 1193.34 | 1195.03 | — | — | — | — | |
| A_25_P00015194 | hsa-miR-1268 | 16459.56 | 15725.31 | 13092.60 | 13009.74 | 3862.85 | 2869.00 | 3177.95 | 3090.57 | 0.22 |
| A_25_P00015195 | hsa-miR-1268 | 19770.13 | 16803.38 | 14066.09 | 13627.82 | 6109.74 | 4557.08 | 4697.63 | 4595.58 | 0.31 |
| A_25_P00015230 | hsa-miR- | 1887.56 | 1927.88 | 1839.01 | 1771.53 | — | — | — | — | |
| | 1274b | | | | | | | | | |
| A_25_P00015231 | hsa-miR- | 2461.53 | 2380.33 | 2408.19 | 2225.83 | 1765.96 | — | 1407.20 | 1384.27 | 0.65 |
| | 1274b | | | | | | | | | |
| A_25_P00015209 | hsa-miR-1275 | 2278.19 | 1885.28 | 1847.10 | 1747.16 | — | — | — | — | |
| A_25_P00015210 | hsa-miR-1275 | 20928.98 | 16442.00 | 15605.04 | 15476.14 | 4156.38 | 3904.41 | 4006.82 | 3444.48 | 0.23 |
| A_25_P00012162 | hsa-miR-128 | 1480.98 | 1462.17 | 1008.33 | 901.17 | — | — | — | — | |
| A_25_P00015239 | hsa-miR-1288 | 2079.08 | 1664.83 | — | — | — | — | — | — | |
| A_25_P00015107 | hsa-miR-1290 | 11072.99 | 7793.28 | 6144.44 | 6097.09 | 3546.88 | 3064.94 | 3312.79 | 2793.78 | 0.41 |
| A_25_P00015133 | hsa-miR-1305 | 4814.10 | 4348.32 | 1784.47 | 1924.95 | — | — | — | — | |
| A_25_P00015134 | hsa-miR-1305 | 4039.18 | 3574.61 | 1612.25 | 1582.14 | — | — | — | — | |
| A_25_P00015249 | hsa-miR-1308 | 8086.66 | 6639.02 | 4031.20 | 3358.41 | — | — | — | — | |
| A_25_P00015250 | hsa-miR-1308 | 22943.53 | 18577.83 | 11143.82 | 9857.05 | — | — | — | — | |
| A_25_P00010439 | hsa-miR-130a | 4480.22 | 3671.84 | 3226.44 | 3198.42 | 1852.05 | 1727.95 | 2042.97 | 1882.56 | 0.51 |
| A_25_P00010440 | hsa-miR-130a | 3049.68 | 2712.43 | 2603.38 | 2462.61 | 1370.58 | 2160.80 | 1833.80 | 1464.24 | 0.63 |
| A_25_P00010437 | hsa-miR-130b | 2143.21 | 1954.47 | 1755.21 | 1718.18 | — | — | — | — | |
| A_25_P00010963 | hsa-miR-133b | 1941.14 | 1829.98 | 1580.06 | 1712.13 | — | — | 1393.11 | — | |
| A_25_P00012230 | hsa-miR-134 | 11828.89 | 9820.55 | 11547.73 | 9986.73 | 4933.75 | 4122.00 | 4455.66 | 3538.98 | 0.39 |
| A_25_P00012231 | hsa-miR-134 | 12295.71 | 10094.00 | 10495.47 | 9139.55 | 4466.45 | 4197.88 | 3842.71 | 3759.98 | 0.39 |
| A_25_P00013406 | hsa-miR- | 4081.86 | 3320.38 | 3298.33 | 3318.56 | 1901.00 | — | 1543.63 | — | 0.49 |
| | 135a* | | | | | | | | | |
| A_25_P00013407 | hsa-miR- | 4886.05 | 4431.99 | 4563.75 | 4442.80 | 2380.33 | 1852.78 | 1642.94 | 1620.35 | 0.41 |
| | 135a* | | | | | | | | | |
| A_25_P00012074 | hsa-miR-139- | 2614.28 | 2345.86 | — | 1274.00 | — | — | — | — | |
| | 3p | | | | | | | | | |
| A_25_P00012176 | hsa-miR-140- | 1419.91 | — | 1514.09 | 1646.89 | — | — | — | — | |
| | 3p | | | | | | | | | |
| A_25_P00012177 | hsa-miR-140- | 3018.34 | 2696.10 | 2908.26 | 2817.82 | — | — | — | — | |
| | 3p | | | | | | | | | |
| A_25_P00011016 | hsa-miR-142- | 2826.21 | 1983.36 | 1358.49 | — | — | — | — | — | |
| | 3p | | | | | | | | | |
| A_25_P00013937 | hsa-miR-142- | 1672.63 | 1388.96 | — | — | — | — | — | — | |
| | 3p | | | | | | | | | |
| A_25_P00014844 | hsa-miR-142- | 1609.72 | 1451.08 | — | 1369.71 | — | — | — | — | |
| | 5p | | | | | | | | | |
| A_25_P00012188 | hsa-miR-144 | 6700.29 | 5025.98 | 2845.03 | 2746.06 | — | — | — | — | |
| A_25_P00012189 | hsa-miR-144 | 8611.71 | 5743.56 | 3364.71 | 3155.81 | — | — | — | — | |
| A_25_P00010078 | hsa-miR-146a | 4730.33 | 4103.21 | 3113.65 | 3101.88 | 2772.98 | 2109.26 | 2832.79 | 2669.23 | 0.69 |
| A_25_P00010079 | hsa-miR-146a | 1509.19 | 1241.16 | 1285.45 | 1461.14 | — | — | — | — | |
| A_25_P00015286 | hsa-miR-1471 | 3351.97 | 3017.41 | 3203.26 | 2972.58 | — | 1096.13 | 1368.88 | 1206.70 | 0.43 |
| A_25_P00015287 | hsa-miR-1471 | 2981.03 | 2767.75 | 2774.29 | 2863.75 | 1199.35 | 2254.03 | — | — | 0.59 |
| A_25_P00010131 | hsa-miR-148a | 1190.32 | 1401.30 | 1145.62 | 1066.57 | — | — | — | — | |
| A_25_P00010132 | hsa-miR-148a | 1833.06 | 1211.48 | 1500.40 | 1392.65 | — | — | — | — | |
| A_25_P00010133 | hsa-miR-148b | 1768.82 | 1353.55 | 1423.13 | 1420.13 | — | — | — | — | |
| A_25_P00013447 | hsa-miR-149* | 1161.70 | — | — | 1307.84 | — | — | — | — | |
| A_25_P00013448 | hsa-miR-149* | 1475.69 | — | 920.26 | 1361.12 | — | — | — | — | |
| A_25_P00013449 | hsa-miR-149* | 2588.03 | 2206.83 | 1983.15 | 1877.08 | 2103.05 | — | — | — | |
| A_25_P00010490 | hsa-miR-150 | 1639.15 | 1553.32 | 1463.14 | 1554.51 | — | — | — | — | |
| A_25_P00014846 | hsa-miR-150 | 6937.20 | 5641.82 | 5021.75 | 5087.59 | — | — | — | — | |
| A_25_P00014847 | hsa-miR-150 | 15575.81 | 14302.65 | 10068.76 | 11380.52 | — | — | — | — | |
| A_25_P00013450 | hsa-miR-150* | 3676.01 | 2929.55 | 5296.48 | 5448.53 | 3748.85 | 2994.56 | 2877.02 | 3041.61 | 0.73 |
| A_25_P00013451 | hsa-miR-150* | 2897.45 | 2671.97 | 4114.02 | 3965.83 | 2446.18 | 1958.17 | 2714.25 | 2193.34 | 0.68 |
| A_25_P00013452 | hsa-miR-150* | 2864.83 | 2607.10 | 3659.33 | 3655.59 | 1810.49 | 1760.80 | 2120.05 | 2380.33 | 0.63 |
| A_25_P00013453 | hsa-miR-150* | 4110.26 | 3254.61 | 4246.84 | 4287.07 | 1877.62 | 2645.76 | 1951.85 | 1989.49 | 0.53 |
| A_25_P00012376 | hsa-miR-151- | 1864.22 | 1750.71 | 1480.46 | 1454.62 | — | — | 1258.79 | — | |
| | 5p | | | | | | | | | |
| A_25_P00010467 | hsa-miR-15a | 16787.82 | 13498.30 | 8772.42 | 8336.53 | 2263.69 | 2109.26 | 2755.82 | 2117.67 | 0.20 |
| A_25_P00014817 | hsa-miR-15a | 38555.57 | 30074.19 | 21557.93 | 21375.55 | 7017.72 | 5328.45 | 5641.82 | 5767.84 | 0.21 |
| A_25_P00011101 | hsa-miR-15b | 35580.93 | 27543.59 | 19340.24 | 19904.83 | 6270.97 | 4720.96 | 4891.37 | 4780.12 | 0.20 |
| A_25_P00011102 | hsa-miR-15b | 9542.93 | 7958.86 | 5208.92 | 5228.68 | 1634.91 | 1278.81 | 1493.47 | 1235.35 | 0.20 |
| A_25_P00010599 | hsa-miR-16 | 23090.50 | 12324.12 | 8655.23 | 10161.87 | 5302.73 | 3759.57 | 4886.03 | 2493.61 | 0.30 |
| A_25_P00014818 | hsa-miR-16 | 11726.85 | 5152.57 | 5600.67 | 6490.04 | 1297.63 | 665.41 | 1059.20 | 892.25 | 0.14 |
| A_25_P00013271 | hsa-miR-16- | 4597.59 | 2821.25 | 2322.62 | 2351.55 | — | — | — | — | |
| | 2* | | | | | | | | | |
| A_25_P00011991 | hsa-miR-17 | 1919.01 | 1772.43 | 1922.02 | 1816.90 | — | — | — | — | |
| A_25_P00013841 | hsa-miR-17 | 6574.66 | 4754.93 | 5075.26 | 4732.36 | — | — | — | — | |
| A_25_P00014819 | hsa-miR-17 | 13988.03 | 13139.22 | 9757.08 | 10654.79 | 1590.60 | — | — | — | |
| A_25_P00010285 | hsa-miR-181a | 1544.36 | 1326.13 | 1055.70 | — | — | — | — | — | |
| A_25_P00014832 | hsa-miR-181a | 1774.66 | 1806.55 | 1477.32 | 1565.46 | — | — | — | — | |
| A_25_P00012098 | hsa-miR-183 | 1471.37 | — | — | 1204.16 | — | — | — | — | |
| A_25_P00012238 | hsa-miR-185 | 4127.08 | 2975.37 | 2802.25 | 2689.72 | — | — | — | — | |
| A_25_P00012239 | hsa-miR-185 | 6422.90 | 6087.57 | 5405.52 | 5151.43 | — | — | — | — | |
| A_25_P00012243 | hsa-miR-186 | 1812.06 | 1629.36 | 1507.49 | 1290.82 | — | — | — | — | |
| A_25_P00013459 | hsa-miR-186* | 1892.32 | 1757.54 | — | — | — | 1195.16 | — | — | |
| A_25_P00013324 | hsa-miR-187* | — | — | 1494.38 | 1638.91 | — | — | 1577.21 | — | |
| A_25_P00013325 | hsa-miR-187* | 1616.16 | 1618.14 | 1910.85 | 1886.39 | 1362.51 | 1473.15 | 1992.60 | 1634.34 | 0.92 |
| A_25_P00013326 | hsa-miR-187* | 1749.03 | 1546.41 | 1701.46 | 1784.88 | — | — | — | — | |
| A_25_P00013327 | hsa-miR-187* | 1852.63 | 1728.90 | 1847.10 | 1803.45 | 1249.34 | — | 1459.93 | — | 0.80 |
| A_25_P00012246 | hsa-miR-188- | 5451.94 | 4605.59 | 4134.62 | 4030.72 | 1974.85 | 1715.65 | 2075.94 | 2039.88 | 0.43 |
| | 5p | | | | | | | | | |
| A_25_P00012247 | hsa-miR-188- | 5788.52 | 4914.99 | 4481.47 | 4114.23 | 2216.35 | 1690.95 | 1975.52 | 1687.09 | 0.39 |
| | 5p | | | | | | | | | |
| A_25_P00013569 | hsa-miR-18b* | 1036.56 | 1374.22 | — | — | — | — | — | — | |
| A_25_P00015315 | hsa-miR- | 1425.04 | 1000.98 | — | — | — | — | — | 866.60 | |
| | 1911* | | | | | | | | | |
| A_25_P00015304 | hsa-miR- | 4246.39 | 3773.70 | 2342.97 | 2391.82 | 1924.58 | 1887.83 | 1522.54 | 1475.58 | 0.53 |
| | 1914* | | | | | | | | | |
| A_25_P00015305 | hsa-miR- | 3319.19 | 3069.81 | 2152.48 | 2109.45 | 1840.78 | 1412.56 | 1888.55 | 1531.19 | 0.63 |
| | 1914* | | | | | | | | | |
| A_25_P00015302 | hsa-miR-1915 | 33665.82 | 33427.92 | 24285.86 | 24101.99 | 7419.60 | 6537.59 | 6895.48 | 6472.84 | 0.24 |
| A_25_P00015303 | hsa-miR-1915 | 39618.34 | 40177.03 | 27089.27 | 25612.34 | 8321.05 | 7134.95 | 5922.13 | 7037.99 | 0.21 |
| A_25_P00010868 | hsa-miR-192 | 1881.22 | 1851.10 | 1619.18 | 1582.14 | — | — | — | — | |
| A_25_P00010869 | hsa-miR-192 | 1339.87 | 1016.27 | — | — | — | — | — | — | |
| A_25_P00013597 | hsa-miR- | 1910.14 | 1523.81 | — | — | — | — | — | — | |
| | 193b* | | | | | | | | | |
| A_25_P00010769 | hsa-miR-195 | 2266.03 | 1846.21 | 1486.64 | 1599.48 | — | — | — | — | |
| A_25_P00012052 | hsa-miR-196a | 1563.64 | 1362.93 | — | — | — | — | — | — | |
| A_25_P00010835 | hsa-miR-197 | 1440.76 | 1682.21 | 1383.84 | 1565.46 | 1381.36 | — | 1316.02 | — | 0.95 |
| A_25_P00012058 | hsa-miR-198 | 1493.01 | — | 1201.16 | 1187.26 | — | — | — | — | |
| A_25_P00012059 | hsa-miR-198 | 2771.61 | 2250.31 | 2237.52 | 2428.89 | — | — | — | — | |
| A_25_P00012063 | hsa-miR- | 1837.88 | 1717.55 | 1389.86 | 1405.68 | 1454.76 | — | 1446.95 | — | 0.94 |
| | 199a-3p | | | | | | | | | |
| A_25_P00012064 | hsa-miR- | 2705.47 | 2041.04 | 1833.28 | 1882.88 | 1886.53 | 1576.68 | — | 1451.49 | 0.78 |
| | 199a-3p | | | | | | | | | |
| A_25_P00010997 | hsa-miR-19a | 10803.90 | 10775.40 | 6690.15 | 6485.16 | 1674.04 | 1591.65 | 1698.35 | 1426.90 | 0.18 |
| A_25_P00010998 | hsa-miR-19a | 7193.29 | 6525.33 | 4081.86 | 4220.43 | — | — | — | — | |
| A_25_P00010999 | hsa-miR-19b | 32110.29 | 34539.31 | 20067.80 | 20610.08 | 4238.15 | 2707.96 | 3608.05 | 4204.67 | 0.14 |
| A_25_P00011000 | hsa-miR-19b | 14799.77 | 11868.80 | 7772.19 | 7195.12 | 1651.73 | 1348.27 | — | 1971.00 | 0.17 |
| A_25_P00010612 | hsa-miR-20a | 22276.33 | 20031.80 | 14344.64 | 14572.25 | 2841.21 | 1372.12 | 1801.73 | 1892.42 | 0.11 |
| A_25_P00010613 | hsa-miR-20a | 14483.73 | 12937.37 | 9256.24 | 10163.12 | 1621.91 | — | 1507.58 | 1301.40 | 0.14 |
| A_25_P00010614 | hsa-miR-20b | 10535.38 | 8579.68 | 6549.16 | 6249.98 | — | — | — | — | |
| A_25_P00010615 | hsa-miR-20b | 3233.25 | 2729.40 | 2495.78 | 2488.70 | — | — | — | — | |
| A_25_P00010975 | hsa-miR-21 | 41195.24 | 54778.63 | 25717.32 | 26869.90 | 12912.91 | 8535.07 | 11054.43 | 10848.91 | 0.29 |
| A_25_P00010976 | hsa-miR-21 | 19131.87 | 16072.42 | 10280.53 | 8539.80 | 5337.48 | 4045.32 | 5022.66 | 5076.20 | 0.36 |
| A_25_P00010204 | hsa-miR-22 | 25276.16 | 25334.53 | 16656.18 | 16206.64 | 9589.20 | 6125.33 | 8602.95 | 9375.45 | 0.40 |
| A_25_P00010205 | hsa-miR-22 | 9325.84 | 7612.29 | 6458.21 | 5863.04 | 3021.87 | 2537.64 | 3519.11 | 3247.01 | 0.42 |
| A_25_P00010690 | hsa-miR-221 | 2484.93 | 2152.10 | 1718.38 | 1630.38 | 1393.94 | 1523.37 | — | — | 0.74 |
| A_25_P00012130 | hsa-miR-223 | 55159.34 | 42864.08 | 20761.73 | 23023.46 | 9111.44 | 6868.03 | 5149.64 | 6051.92 | 0.19 |
| A_25_P00012131 | hsa-miR-223 | 6307.73 | 5659.90 | 3190.24 | 4016.76 | 2338.23 | 1287.98 | 705.99 | 829.67 | 0.27 |
| A_25_P00010843 | hsa-miR-23a | 5909.30 | 4851.15 | 4392.02 | 4248.46 | 3290.22 | 2341.20 | 3078.81 | 2939.34 | 0.60 |
| A_25_P00014820 | hsa-miR-23a | 17587.72 | 15432.58 | 12310.75 | 11870.67 | 8662.82 | 6319.01 | 6133.43 | 7506.96 | 0.50 |
| A_25_P00010881 | hsa-miR-23b | 1364.82 | 1082.62 | — | 1465.80 | — | — | — | — | |
| A_25_P00010676 | hsa-miR-24 | 6148.67 | 6002.10 | 4644.62 | 4517.29 | 3471.76 | 1906.08 | 2926.53 | 2838.77 | 0.52 |
| A_25_P00010677 | hsa-miR-24 | 5598.03 | 5356.04 | 4284.01 | 4173.66 | 3222.97 | 1931.24 | 2661.18 | 2598.88 | 0.54 |
| A_25_P00010989 | hsa-miR-25 | 37300.08 | 36473.75 | 28948.43 | 28754.63 | 4773.71 | 3474.74 | 4357.48 | 4948.49 | 0.13 |
| A_25_P00010990 | hsa-miR-25 | 16149.80 | 13715.62 | 12788.39 | 12597.53 | 1827.81 | — | 1732.76 | 1810.73 | 0.14 |
| A_25_P00011998 | hsa-miR-26a | 1600.43 | — | — | 841.10 | — | — | — | — | |
| A_25_P00011999 | hsa-miR-26a | 2913.61 | 2504.67 | 1777.29 | 1755.21 | 1606.89 | — | — | 1111.36 | 0.68 |
| A_25_P00012001 | hsa-miR-26b | 9147.38 | 7020.54 | 4219.11 | 4081.86 | — | — | — | — | |
| A_25_P00012002 | hsa-miR-26b | 11962.35 | 10296.38 | 6071.32 | 6163.54 | 1545.13 | 1061.92 | — | — | 0.20 |
| A_25_P00010797 | hsa-miR-27a | 2497.82 | 2052.05 | 2034.14 | 2038.33 | 1476.09 | — | — | 1514.20 | 0.72 |
| A_25_P00014821 | hsa-miR-27a | 5081.66 | 4540.98 | 3604.13 | 3294.24 | 2531.30 | 1826.11 | 2521.11 | 2152.94 | 0.55 |
| A_25_P00010761 | hsa-miR-27b | 1452.80 | — | — | 1322.99 | — | — | — | — | |
| A_25_P00014837 | hsa-miR-27b | 2294.59 | 1765.12 | 1885.80 | 1891.76 | 2137.49 | 1610.38 | 1757.99 | 1676.12 | 0.92 |
| A_25_P00013978 | hsa-miR-296- | — | — | 1473.74 | 1536.83 | — | — | — | — | |
| | 5p | | | | | | | | | |
| A_25_P00012012 | hsa-miR-29a | 861.17 | — | 1467.46 | — | 2495.42 | — | — | — | |
| A_25_P00012013 | hsa-miR-29a | 3928.83 | 2821.25 | 2623.11 | 2651.16 | 1510.95 | 1327.40 | 1612.11 | 1266.36 | 0.48 |
| A_25_P00010053 | hsa-miR-29b | 1397.54 | 1335.22 | — | — | — | — | — | — | |
| A_25_P00012274 | hsa-miR-29c | 3196.00 | 2306.25 | 2100.61 | 2012.98 | 1433.64 | 1398.58 | — | — | 0.63 |
| A_25_P00012275 | hsa-miR-29c | 3809.34 | 2747.05 | 2512.35 | 2332.81 | — | — | 1422.40 | — | |
| A_25_P00010839 | hsa-miR-301a | 1664.94 | 1437.38 | — | — | — | — | — | — | |
| A_25_P00014838 | hsa-miR-30b | 1677.49 | 1441.86 | — | 1353.43 | — | — | — | — | |
| A_25_P00013489 | hsa-miR-30c- | 1371.74 | 1231.15 | 1671.04 | 1763.35 | — | — | — | — | |
| | 1* | | | | | | | | | |
| A_25_P00010682 | hsa-miR-30d | 5038.42 | 4966.31 | 4720.96 | 4350.14 | 1720.36 | — | 1560.59 | 1860.95 | 0.37 |
| A_25_P00010683 | hsa-miR-30d | 4275.98 | 3956.16 | 3705.46 | 3479.28 | 1143.09 | 1495.01 | 1680.12 | — | 0.39 |
| A_25_P00012300 | hsa-miR-30e | 1553.98 | 1394.64 | 1556.55 | 1257.47 | — | — | — | — | |
| A_25_P00012301 | hsa-miR-30e | 3587.89 | 2907.69 | 2824.38 | 2708.46 | — | — | — | 993.75 | |
| A_25_P00012261 | hsa-miR-320a | 2333.56 | 2137.01 | 3165.39 | 3054.23 | 1524.89 | — | — | — | |
| A_25_P00012262 | hsa-miR-320a | 4404.56 | 3298.17 | 6223.37 | 6004.16 | 1575.71 | — | 1757.99 | 1726.96 | 0.35 |
| A_25_P00015034 | hsa-miR-320b | 2245.39 | 2083.53 | 3029.78 | 3019.11 | — | — | — | — | |
| A_25_P00015035 | hsa-miR-320b | 7877.66 | 6805.61 | 7949.97 | 8058.85 | 3644.61 | 2202.84 | 2789.34 | 2500.96 | 0.36 |
| A_25_P00015036 | hsa-miR-320c | 58697.63 | 37632.37 | 41712.36 | 39530.19 | 11635.93 | 10231.01 | 9975.50 | 9889.64 | 0.24 |
| A_25_P00015037 | hsa-miR-320c | — | 58697.63 | 45295.50 | 41566.22 | 11999.23 | 9885.12 | 9573.14 | 10262.05 | 0.22 |
| A_25_P00015270 | hsa-miR-320d | 6016.13 | 5873.46 | 4900.55 | 4910.85 | 2881.91 | 1999.47 | 2248.94 | 2543.58 | 0.45 |
| A_25_P00015271 | hsa-miR-320d | 13011.90 | 14042.91 | 11939.00 | 11705.41 | 4014.11 | 2799.07 | 3405.47 | 3894.08 | 0.28 |
| A_25_P00010539 | hsa-miR-324- | 1884.48 | 1906.08 | 1570.87 | 1679.18 | — | — | — | — | |
| | 3p | | | | | | | | | |
| A_25_P00010540 | hsa-miR-324- | 1526.49 | — | 1025.77 | 1029.92 | — | — | — | — | |
| | 3p | | | | | | | | | |
| A_25_P00012396 | hsa-miR-338- | — | 897.89 | 1640.26 | — | — | — | — | — | |
| | 3p | | | | | | | | | |
| A_25_P00012402 | hsa-miR-339- | 2001.86 | 1489.36 | — | 1991.60 | — | — | — | — | |
| | 3p | | | | | | | | | |
| A_25_P00012403 | hsa-miR-339- | 1653.23 | 1514.01 | — | 1082.51 | — | — | — | — | |
| | 3p | | | | | | | | | |
| A_25_P00012404 | hsa-miR-339- | 1714.06 | 1482.27 | — | — | — | — | — | — | |
| | 3p | | | | | | | | | |
| A_25_P00012357 | hsa-miR-342- | 1178.46 | 1623.76 | 1376.36 | 1426.23 | — | — | — | — | |
| | 3p | | | | | | | | | |
| A_25_P00012358 | hsa-miR-342- | 3299.68 | 2886.76 | 2670.72 | 2726.70 | — | — | — | — | |
| | 3p | | | | | | | | | |
| A_25_P00010953 | hsa-miR-363 | 5262.75 | 4228.33 | 3484.83 | 3518.51 | — | — | — | — | |
| A_25_P00010954 | hsa-miR-363 | 3217.47 | 2490.92 | 2262.54 | 2179.48 | — | — | — | — | |
| A_25_P00013991 | hsa-miR-369- | — | — | — | — | — | 666.22 | — | 1074.82 | |
| | 3p | | | | | | | | | |
| A_25_P00012323 | hsa-miR-371- | 1589.21 | — | 1528.99 | 1333.03 | — | — | — | — | |
| | 5p | | | | | | | | | |
| A_25_P00012324 | hsa-miR-371- | 1118.69 | — | 1396.23 | 1115.42 | — | — | — | — | |
| | 5p | | | | | | | | | |
| A_25_P00012418 | hsa-miR-423- | 1448.42 | — | 1677.33 | 1624.12 | — | — | — | — | |
| | 5p | | | | | | | | | |
| A_25_P00012419 | hsa-miR-423- | 3261.31 | 2469.88 | 3522.71 | 3413.13 | 1497.44 | — | — | — | |
| | 5p | | | | | | | | | |
| A_25_P00011109 | hsa-miR-424 | 1755.21 | 1588.84 | — | 1231.03 | — | — | — | — | |
| A_25_P00010977 | hsa-miR-425 | 5683.42 | 4683.19 | 3967.45 | 3885.39 | — | — | — | — | |
| A_25_P00014045 | hsa-miR-425 | 3082.05 | 2523.15 | 2532.06 | 2503.81 | — | — | — | — | |
| A_25_P00012446 | hsa-miR-451 | Saturation | Saturation | Saturation | Saturation | 9787.84 | 9268.96 | 9094.77 | 6829.93 | |
| A_25_P00012459 | hsa-miR-483- | 13506.07 | 12092.45 | 10809.71 | 10416.88 | 2799.67 | 2380.33 | 2596.29 | 2452.37 | 0.22 |
| | 5p | | | | | | | | | |
| A_25_P00014861 | hsa-miR-483- | 20355.81 | 17717.45 | 14693.80 | 14258.34 | 5053.24 | 4266.79 | 4535.84 | 4277.58 | 0.27 |
| | 5p | | | | | | | | | |
| A_25_P00010431 | hsa-miR-484 | 1960.49 | 1560.59 | 2023.26 | 2026.51 | — | — | — | — | |
| A_25_P00010432 | hsa-miR-484 | — | — | 1451.27 | 1439.40 | — | — | — | — | |
| A_25_P00014063 | hsa-miR-486- | 8932.75 | 7344.23 | 6490.04 | 7509.94 | 1773.76 | 788.28 | 1549.16 | 1706.10 | 0.16 |
| | 5p | | | | | | | | | |
| A_25_P00014064 | hsa-miR-486- | 3914.86 | 4746.15 | 4581.74 | 5059.55 | 717.52 | 614.80 | 764.03 | 769.61 | 0.19 |
| | 5p | | | | | | | | | |
| A_25_P00010688 | hsa-miR-498 | 1681.05 | 1255.10 | — | — | — | — | — | — | |
| A_25_P00012624 | hsa-miR-499- | 1077.91 | — | 946.91 | — | 1223.95 | — | 1476.18 | — | 1.13 |
| | 5p | | | | | | | | | |
| A_25_P00012625 | hsa-miR-499- | 1444.06 | 1211.48 | — | — | — | — | 1358.97 | 1440.89 | 1.01 |
| | 5p | | | | | | | | | |
| A_25_P00014918 | hsa-miR-509- | 1986.67 | 1838.30 | 1182.93 | 1439.40 | — | — | — | — | |
| | 3-5p | | | | | | | | | |
| A_25_P00012660 | hsa-miR- | 1052.51 | 1466.23 | — | — | — | — | — | — | |
| | 513a-3p | | | | | | | | | |
| A_25_P00012618 | hsa-miR- | 1331.00 | — | 1972.98 | — | — | — | — | — | |
| | 516a-5p | | | | | | | | | |
| A_25_P00014151 | hsa-miR- | 3392.88 | 3228.95 | 1882.17 | 1938.29 | — | — | — | — | |
| | 516a-5p | | | | | | | | | |
| A_25_P00010563 | hsa-miR-520b | 1520.87 | 1143.87 | — | — | — | — | — | — | |
| A_25_P00012692 | hsa-miR-532- | — | 670.31 | 898.92 | — | — | — | — | — | |
| | 3p | | | | | | | | | |
| A_25_P00010344 | hsa-miR-557 | 1693.99 | 1425.30 | 1363.65 | 1479.12 | — | — | — | — | |
| A_25_P00010345 | hsa-miR-557 | 1874.76 | — | 1267.82 | — | — | — | — | — | |
| A_25_P00011096 | hsa-miR-572 | 8932.92 | 8716.79 | 8609.28 | 7834.06 | 1180.04 | — | — | — | |
| A_25_P00011097 | hsa-miR-572 | 1258.29 | — | 1539.94 | — | — | — | — | — | |
| A_25_P00012724 | hsa-miR-574- | 2401.47 | 1736.22 | 2785.41 | 2781.46 | 5895.82 | 3552.57 | 9049.71 | 4489.75 | 2.37 |
| | 5p | | | | | | | | | |
| A_25_P00012725 | hsa-miR-574- | 4507.02 | 2540.08 | 4345.26 | 3702.58 | 7831.25 | 5912.86 | 10428.21 | 6222.09 | 2.01 |
| | 5p | | | | | | | | | |
| A_25_P00010808 | hsa-miR-575 | 7370.09 | 6445.98 | 4819.29 | 4394.78 | — | — | — | — | |
| A_25_P00014896 | hsa-miR-575 | 7574.04 | 6915.84 | 5139.00 | 4835.06 | — | — | — | — | |
| A_25_P00010891 | hsa-miR-583 | 2065.00 | 1793.69 | — | — | — | — | — | — | |
| A_25_P00010892 | hsa-miR-583 | 2545.08 | 2169.56 | 1457.41 | — | — | 1808.17 | — | — | |
| A_25_P00010634 | hsa-miR-584 | 1286.30 | — | 1112.20 | — | — | — | — | — | |
| A_25_P00010640 | hsa-miR-601 | 4670.72 | 4147.94 | 1794.68 | 1839.77 | — | — | — | — | |
| A_25_P00010641 | hsa-miR-601 | 3450.20 | 3539.43 | 1898.73 | 1700.52 | — | — | — | — | |
| A_25_P00010642 | hsa-miR-601 | 3634.29 | 3467.86 | 1935.19 | 1808.76 | — | — | — | — | |
| A_25_P00010643 | hsa-miR-601 | 2842.98 | 4124.56 | 1959.54 | 2049.87 | — | — | — | — | |
| A_25_P00010805 | hsa-miR-622 | 1486.23 | 1292.34 | 1875.33 | 1962.31 | — | — | — | — | |
| A_25_P00010806 | hsa-miR-622 | 1705.92 | — | 2119.69 | 2067.03 | — | — | — | — | |
| A_25_P00010807 | hsa-miR-622 | 1568.78 | 1368.66 | 2078.21 | 1975.95 | — | — | — | — | |
| A_25_P00010226 | hsa-miR-623 | 1146.94 | 1269.05 | — | 1343.47 | — | — | — | — | |
| A_25_P00010227 | hsa-miR-623 | 3768.51 | 3051.49 | 2714.75 | 2543.21 | — | — | — | — | |
| A_25_P00010228 | hsa-miR-623 | 1899.87 | 1688.01 | 1747.28 | 1795.19 | — | — | — | — | |
| A_25_P00010229 | hsa-miR-623 | 1980.89 | 1678.27 | 1816.30 | 1617.97 | — | — | — | — | |
| A_25_P00010248 | hsa-miR-630 | — | — | 58697.63 | 54383.61 | 10359.80 | 9391.53 | 8159.64 | 8845.49 | 0.19 |
| A_25_P00010249 | hsa-miR-630 | — | — | 54428.09 | 58697.63 | 9962.54 | 8886.43 | 7706.92 | 8500.78 | 0.18 |
| A_25_P00010402 | hsa-miR-638 | 12537.73 | 6451.63 | 5059.55 | 5600.67 | 4237.13 | 4987.31 | 2385.92 | 1985.11 | 0.46 |
| A_25_P00010403 | hsa-miR-638 | 51388.09 | 50575.71 | 31736.55 | 29844.41 | 10901.74 | 8025.66 | 6348.13 | 6764.52 | 0.20 |
| A_25_P00012834 | hsa-miR-652 | 1845.44 | 1593.59 | 1308.95 | 1307.84 | — | — | — | — | |
| A_25_P00010459 | hsa-miR-660 | 1553.98 | 1478.56 | 1371.66 | 1476.23 | — | — | — | — | |
| A_25_P00010799 | hsa-miR-663 | 10362.67 | 10492.30 | 13259.60 | 13393.78 | — | — | — | — | |
| A_25_P00010800 | hsa-miR-663 | 4224.00 | 3623.73 | 5824.41 | 5721.62 | — | — | — | — | |
| A_25_P00013004 | hsa-miR-665 | 1225.56 | 940.74 | — | — | — | — | — | — | |
| A_25_P00012860 | hsa-miR-671- | 2936.63 | 2626.13 | 2476.65 | 2371.75 | 2010.31 | 1742.98 | 1815.77 | 1830.06 | 0.71 |
| | 5p | | | | | | | | | |
| A_25_P00012861 | hsa-miR-671- | 4444.95 | 3819.02 | 2985.43 | 2879.20 | 1778.73 | 1678.27 | 1476.18 | 1761.06 | 0.47 |
| | 5p | | | | | | | | | |
| A_25_P00012078 | hsa-miR-7 | 1626.30 | — | — | 1471.32 | — | — | — | — | |
| A_25_P00012971 | hsa-miR-708 | 1778.81 | 1495.71 | — | — | — | — | — | — | |
| A_25_P00015264 | hsa-miR-720 | — | — | — | — | 1560.59 | 1131.43 | — | 2289.49 | |
| A_25_P00015265 | hsa-miR-720 | 3160.42 | 2561.40 | 3251.15 | 3223.63 | 5171.38 | 3659.33 | 4106.70 | 4393.52 | 1.42 |
| A_25_P00011341 | hsa-miR-765 | 2186.03 | 2019.36 | 1631.37 | 1526.11 | — | — | — | — | |
| A_25_P00011342 | hsa-miR-765 | 5330.33 | 4504.49 | 3059.04 | 2768.80 | 1406.75 | — | — | — | |
| A_25_P00011232 | hsa-miR-769- | 1271.76 | 1456.59 | 2932.70 | 2837.84 | — | — | — | — | |
| | 3p | | | | | | | | | |
| A_25_P00012918 | hsa-miR-874 | 1645.76 | 1431.14 | — | — | — | — | — | — | |
| A_25_P00012919 | hsa-miR-874 | 1735.89 | 1881.85 | 1329.19 | 1449.00 | — | — | — | — | |
| A_25_P00012997 | hsa-miR-877 | 1414.65 | 1471.44 | 1096.13 | — | — | — | — | — | |
| A_25_P00012998 | hsa-miR-877 | 1740.95 | 1507.91 | 1055.70 | 1242.29 | — | — | — | — | |
| A_25_P00012999 | hsa-miR-877 | 1685.28 | 1611.61 | 1659.56 | 1505.54 | — | — | — | — | |
| A_25_P00013001 | hsa-miR-887 | 2021.91 | 1916.38 | — | — | — | — | — | — | |
| A_25_P00012927 | hsa-miR-891b | 806.71 | 1601.58 | — | — | — | — | — | — | |
| A_25_P00013050 | hsa-miR-923 | 15307.00 | 14563.76 | 7509.94 | 8655.23 | 7126.56 | 6620.71 | 6458.55 | 5115.59 | 0.55 |
| A_25_P00012030 | hsa-miR-92a | 44095.86 | 46336.34 | 34753.18 | 33433.28 | 6776.33 | 5641.82 | 6566.18 | 7939.36 | 0.17 |
| A_25_P00012031 | hsa-miR-92a | 5612.61 | 4270.14 | 4016.76 | 4581.74 | 1185.51 | 734.70 | 891.64 | 1330.90 | 0.22 |
| A_25_P00010610 | hsa-miR-93 | 13318.34 | 11448.21 | 9926.59 | 10978.79 | 1953.27 | 1661.11 | 1780.97 | 1567.67 | 0.15 |
| A_25_P00010611 | hsa-miR-93 | 2511.98 | 2029.10 | 2579.95 | 2567.14 | — | — | — | — | |
| A_25_P00013074 | hsa-miR-936 | 2791.84 | 2185.32 | 2171.70 | 2269.72 | — | — | — | — | |
| A_25_P00013086 | hsa-miR-939 | 13696.81 | 11724.10 | 7565.71 | 6900.10 | 1465.75 | 1455.71 | — | — | 0.18 |
| A_25_P00013087 | hsa-miR-939 | 23825.81 | 20669.21 | 15093.11 | 14966.81 | 3074.04 | 2449.56 | 2155.42 | 1945.69 | 0.13 |
| A_25_P00013089 | hsa-miR-940 | 1621.59 | 1419.63 | 1825.11 | 1900.92 | — | 22421.07 | — | — | |
| A_25_P00013090 | hsa-miR-940 | 2660.57 | 2327.78 | 3077.13 | 2903.48 | 1443.95 | 1480.46 | — | 1398.33 | 0.55 |
| A_25_P00012035 | hsa-miR-96 | 1458.34 | 1067.99 | 1318.79 | 986.69 | — | — | — | — | |

TABLE 6
List of 13 remodelling-associated proteins (=seed proteins = remodelling
proteins) and their 26 interacting proteins (=interactor).

| Seed | Interactor |
|---|---|
| ADRB1 | MAGI2 |
| CRP | FCGR2A |
| CRP | FCGR2C |
| HGF | HGFAC |
| HGF | MET |
| MDK | RPL18A |
| MDK | UBQLN4 |
| MMP2 | HSP90AA1 |
| MMP2 | LAMC2 |
| MMP2 | TIMP2 |
| MMP9 | ITGA5 |
| MMP9 | LCN2 |
| NOS3 | ESR1 |
| NPPA | UBQLN4 |
| NPPB | EWSR1 |
| STUB1 | AKT1 |
| STUB1 | HSPA8 |
| STUB1 | KHDRBS1 |
| STUB1 | MAP3K14 |
| STUB1 | MAP3K3 |
| STUB1 | MAPT |
| STUB1 | MPP1 |
| STUB1 | TRAF2 |
| TFAM | IKBKE |
| TFAM | MCC |
| TFAM | TFB2M |
| TFAM | TNIK |

TABLE 7
The 265 miRNAs targeting both seed proteins and their interactors

| Gene | miRNA |
|---|---|
| ACE | hsa-miR-890 |
| ACE | hsa-miR-138 |
| ACE | hsa-miR-199b-5p |
| ACE | hsa-miR-22 |
| ACE | hsa-miR-24 |
| ACE | hsa-miR-27a |
| ACE | hsa-miR-323-5p |
| ACE | hsa-miR-432 |
| ACE | hsa-miR-551a |
| ACE | hsa-miR-593 |
| ACE | hsa-miR-635 |
| ACE | hsa-miR-876-3p |
| ADRB1 | hsa-miR-937 |
| ADRB1 | hsa-miR-101 |
| ADRB1 | hsa-miR-141 |
| ADRB1 | hsa-miR-142-3p |
| ADRB1 | hsa-miR-150 |
| ADRB1 | hsa-miR-188-5p |
| ADRB1 | hsa-miR-30a |
| ADRB1 | hsa-miR-30b |
| ADRB1 | hsa-miR-30c |
| ADRB1 | hsa-miR-30d |
| ADRB1 | hsa-miR-331-3p |
| ADRB1 | hsa-miR-526b |
| ADRB1 | hsa-miR-578 |
| ADRB1 | hsa-miR-671-5p |
| ADRB1 | hsa-miR-891a |
| AKT1 | hsa-miR-885-3p |
| AKT1 | hsa-miR-138 |
| AKT1 | hsa-miR-409-3p |
| AKT1 | hsa-miR-501-3p |
| AKT1 | hsa-miR-655 |
| CRP | hsa-miR-939 |
| CRP | hsa-miR-10a |
| CRP | hsa-miR-133a |
| CRP | hsa-miR-146b-3p |
| CRP | hsa-miR-150 |
| CRP | hsa-miR-186 |
| CRP | hsa-miR-27a |
| CRP | hsa-miR-299-3p |
| CRP | hsa-miR-323-5p |
| CRP | hsa-miR-362-5p |
| CRP | hsa-miR-424 |
| CRP | hsa-miR-500 |
| CRP | hsa-miR-542-5p |
| CRP | hsa-miR-609 |
| CRP | hsa-miR-624 |
| CRP | hsa-miR-631 |
| CRP | hsa-miR-661 |
| CRP | hsa-miR-7 |
| CRP | hsa-miR-802 |
| CRP | hsa-miR-920 |
| ESR1 | hsa-miR-934 |
| ESR1 | hsa-miR-148a |
| ESR1 | hsa-miR-18a |
| ESR1 | hsa-miR-19a |
| ESR1 | hsa-miR-204 |
| ESR1 | hsa-miR-22 |
| ESR1 | hsa-miR-222 |
| ESR1 | hsa-miR-26a |
| ESR1 | hsa-miR-324-3p |
| ESR1 | hsa-miR-33a |
| ESR1 | hsa-miR-34b |
| ESR1 | hsa-miR-650 |
| ESR1 | hsa-miR-9 |
| EWSR1 | hsa-miR-768-3p |
| EWSR1 | hsa-miR-299-5p |
| EWSR1 | hsa-miR-409-3p |
| EWSR1 | hsa-miR-522 |
| EWSR1 | hsa-miR-582-5p |
| EWSR1 | hsa-miR-593 |
| EWSR1 | hsa-miR-659 |
| FCGR2A | hsa-miR-643 |
| FCGR2A | hsa-miR-297 |
| FCGR2A | hsa-miR-331-5p |
| FCGR2A | hsa-miR-337-5p |
| FCGR2A | hsa-miR-512-5p |
| FCGR2A | hsa-miR-581 |
| FCGR2A | hsa-miR-640 |
| FCGR2C | hsa-miR-331-5p |
| HGF | hsa-miR-522 |
| HGF | hsa-let-7d |
| HGF | hsa-miR-190 |
| HGFAC | hsa-miR-940 |
| HSP90AA1 | hsa-miR-888 |
| HSP90AA1 | hsa-miR-146b-3p |
| HSP90AA1 | hsa-miR-148a |
| HSP90AA1 | hsa-miR-196a |
| HSP90AA1 | hsa-miR-219-2-3p |
| HSP90AA1 | hsa-miR-362-5p |
| HSP90AA1 | hsa-miR-411 |
| HSP90AA1 | hsa-miR-518d-5p |
| HSP90AA1 | hsa-miR-550 |
| HSP90AA1 | hsa-miR-591 |
| HSPA8 | hsa-miR-646 |
| HSPA8 | hsa-miR-142-5p |
| HSPA8 | hsa-miR-147 |
| HSPA8 | hsa-miR-222 |
| HSPA8 | hsa-miR-26a |
| HSPA8 | hsa-miR-301a |
| HSPA8 | hsa-miR-26a |
| HSPA8 | hsa-miR-301a |
| HSPA8 | hsa-miR-338-5p |
| HSPA8 | hsa-miR-33a |
| HSPA8 | hsa-miR-340 |
| HSPA8 | hsa-miR-365 |
| HSPA8 | hsa-miR-448 |
| HSPA8 | hsa-miR-499-5p |
| HSPA8 | hsa-miR-519a |
| HSPA8 | hsa-miR-519d |
| HSPA8 | hsa-miR-580 |
| HSPA8 | hsa-miR-587 |
| HSPA8 | hsa-miR-590-3p |
| HSPA8 | hsa-miR-641 |
| IKBKE | hsa-miR-769-3p |
| IKBKE | hsa-let-7b |
| IKBKE | hsa-let-7c |
| IKBKE | hsa-miR-155 |
| IKBKE | hsa-miR-192 |
| IKBKE | hsa-miR-24 |
| IKBKE | hsa-miR-455-5p |
| IKBKE | hsa-miR-485-5p |
| IKBKE | hsa-miR-492 |
| IKBKE | hsa-miR-576-5p |
| IKBKE | hsa-miR-604 |
| IKBKE | hsa-miR-7 |
| ITGA5 | hsa-miR-936 |
| ITGA5 | hsa-miR-148a |
| ITGA5 | hsa-miR-149 |
| ITGA5 | hsa-miR-25 |
| ITGA5 | hsa-miR-26a |
| ITGA5 | hsa-miR-27a |
| ITGA5 | hsa-miR-296-3p |
| ITGA5 | hsa-miR-30b |
| ITGA5 | hsa-miR-32 |
| ITGA5 | hsa-miR-328 |
| ITGA5 | hsa-miR-338-3p |
| ITGA5 | hsa-miR-367 |
| ITGA5 | hsa-miR-382 |
| ITGA5 | hsa-miR-384 |
| ITGA5 | hsa-miR-432 |
| ITGA5 | hsa-miR-486-3p |
| ITGA5 | hsa-miR-515-5p |
| ITGA5 | hsa-miR-760 |
| ITGA5 | hsa-miR-876-5p |
| KHDRBS1 | hsa-miR-662 |
| KHDRBS1 | hsa-miR-200b |
| KHDRBS1 | hsa-miR-203 |
| KHDRBS1 | hsa-miR-204 |
| KHDRBS1 | hsa-miR-218 |
| KHDRBS1 | hsa-miR-302c* |
| LAMC2 | hsa-miR-767-5p |
| LAMC2 | hsa-miR-1 |
| LAMC2 | hsa-miR-124 |
| LAMC2 | hsa-miR-193b |
| LAMC2 | hsa-miR-23a |
| LAMC2 | hsa-miR-323-3p |
| LAMC2 | hsa-miR-548b-3p |
| LAMC2 | hsa-miR-615-3p |
| LAMC2 | hsa-miR-660 |
| LCN2 | hsa-miR-939 |
| LCN2 | hsa-miR-138 |
| LCN2 | hsa-miR-491-5p |
| LCN2 | hsa-miR-608 |
| LCN2 | hsa-miR-646 |
| LCN2 | hsa-miR-675 |
| LCN2 | hsa-miR-923 |
| MAGI2 | hsa-miR-887 |
| MAGI2 | hsa-miR-1 |
| MAGI2 | hsa-miR-101 |
| MAGI2 | hsa-miR-134 |
| MAGI2 | hsa-miR-141 |
| MAGI2 | hsa-miR-142-5p |
| MAGI2 | hsa-miR-144 |
| MAGI2 | hsa-miR-19a |
| MAGI2 | hsa-miR-218 |
| MAGI2 | hsa-miR-22 |
| MAGI2 | hsa-miR-221 |
| MAGI2 | hsa-miR-27a |
| MAGI2 | hsa-miR-28-3p |
| MAGI2 | hsa-miR-542-3p |
| MAGI2 | hsa-miR-556-5p |
| MAGI2 | hsa-miR-587 |
| MAGI2 | hsa-miR-610 |
| MAGI2 | hsa-miR-629 |
| MAGI2 | hsa-miR-651 |
| MAP3K14 | hsa-miR-892a |
| MAP3K14 | hsa-miR-137 |
| MAP3K14 | hsa-miR-155 |
| MAP3K14 | hsa-miR-19a |
| MAP3K14 | hsa-miR-27a |
| MAP3K14 | hsa-miR-31 |
| MAP3K14 | hsa-miR-372 |
| MAP3K14 | hsa-miR-412 |
| MAP3K14 | hsa-miR-492 |
| MAP3K14 | hsa-miR-514 |
| MAP3K14 | hsa-miR-517a |
| MAP3K14 | hsa-miR-621 |
| MAP3K14 | hsa-miR-630 |
| MAP3K14 | hsa-miR-634 |
| MAP3K14 | hsa-miR-665 |
| MAP3K14 | hsa-miR-874 |
| MAP3K3 | hsa-miR-96 |
| MAP3K3 | hsa-let-7b |
| MAP3K3 | hsa-miR-103 |
| MAP3K3 | hsa-miR-133a |
| MAP3K3 | hsa-miR-141 |
| MAP3K3 | hsa-miR-145 |
| MAP3K3 | hsa-miR-181a |
| MAP3K3 | hsa-miR-182 |
| MAP3K3 | hsa-miR-193b |
| MAP3K3 | hsa-miR-194 |
| MAP3K3 | hsa-miR-204 |
| MAP3K3 | hsa-miR-206 |
| MAP3K3 | hsa-miR-22 |
| MAP3K3 | hsa-miR-23a |
| MAP3K3 | hsa-miR-891b |
| MAP3K3 | hsa-miR-9 |
| MAP3K3 | hsa-miR-93 |
| MAPT | hsa-miR-563 |
| MAPT | hsa-miR-34a |
| MCC | hsa-miR-628-5p |
| MCC | hsa-miR-136 |
| MCC | hsa-miR-215 |
| MCC | hsa-miR-24 |
| MCC | hsa-miR-296-3p |
| MCC | hsa-miR-371-3p |
| MCC | hsa-miR-450b-3p |
| MCC | hsa-miR-450b-5p |
| MCC | hsa-miR-501-3p |
| MCC | hsa-miR-600 |
| MCC | hsa-miR-605 |
| MCC | hsa-miR-625 |
| MDK | hsa-miR-940 |
| MDK | hsa-miR-124 |
| MDK | hsa-miR-188-5p |
| MDK | hsa-miR-219-2-3p |
| MDK | hsa-miR-223 |
| MDK | hsa-miR-23a |
| MDK | hsa-miR-326 |
| MDK | hsa-miR-491-5p |
| MDK | hsa-miR-608 |
| MDK | hsa-miR-623 |
| MDK | hsa-miR-625 |
| MDK | hsa-miR-760 |
| MET | hsa-miR-876-3p |
| MET | hsa-miR-1 |
| MET | hsa-miR-101 |
| MET | hsa-miR-122 |
| MET | hsa-miR-130a |
| MET | hsa-miR-133a |
| MET | hsa-miR-144 |
| MET | hsa-miR-182 |
| MET | hsa-miR-186 |
| MET | hsa-miR-198 |
| MET | hsa-miR-23a |
| MET | hsa-miR-31 |
| MET | hsa-miR-335 |
| MET | hsa-miR-337-3p |
| MET | hsa-miR-338-5p |
| MET | hsa-miR-34a |
| MET | hsa-miR-34b |
| MET | hsa-miR-34c-5p |
| MET | hsa-miR-369-3p |
| MET | hsa-miR-374b |
| MET | hsa-miR-381 |
| MET | hsa-miR-509-3p |
| MET | hsa-miR-520g |
| MET | hsa-miR-548d-3p |
| MET | hsa-miR-595 |
| MET | hsa-miR-616 |
| MET | hsa-miR-633 |
| MMP2 | hsa-miR-644 |
| MMP2 | hsa-miR-136 |
| MMP2 | hsa-miR-299-3p |
| MMP2 | hsa-miR-29a |
| MMP2 | hsa-miR-486-3p |
| MMP2 | hsa-miR-519e |
| MMP2 | hsa-miR-564 |
| MMP3 | hsa-miR-874 |
| MMP3 | hsa-miR-146b-3p |
| MMP3 | hsa-miR-15b |
| MMP3 | hsa-miR-17 |
| MMP3 | hsa-miR-18a |
| MMP3 | hsa-miR-204 |
| MMP3 | hsa-miR-27a |
| MMP3 | hsa-miR-31 |
| MMP3 | hsa-miR-365 |
| MMP3 | hsa-miR-516a-3p |
| MMP3 | hsa-miR-520f |
| MMP3 | hsa-miR-542-3p |
| MMP3 | hsa-miR-574-3p |
| MMP3 | hsa-miR-574-5p |
| MMP3 | hsa-miR-577 |
| MMP3 | hsa-miR-623 |
| MMP3 | hsa-miR-624 |
| MMP9 | hsa-miR-892b |
| MMP9 | hsa-miR-133a |
| MMP9 | hsa-miR-149 |
| MMP9 | hsa-miR-183 |
| MMP9 | hsa-miR-204 |
| MMP9 | hsa-miR-296-3p |
| MMP9 | hsa-miR-330-3p |
| MMP9 | hsa-miR-483-3p |
| MMP9 | hsa-miR-491-5p |
| MPP1 | hsa-miR-892b |
| MPP1 | hsa-miR-105 |
| MPP1 | hsa-miR-137 |
| MPP1 | hsa-miR-296-5p |
| MPP1 | hsa-miR-363 |
| MPP1 | hsa-miR-423-5p |
| MPP1 | hsa-miR-500 |
| MPP1 | hsa-miR-501-5p |
| MPP1 | hsa-miR-515-5p |
| MPP1 | hsa-miR-518d-5p |
| MPP1 | hsa-miR-576-3p |
| MPP1 | hsa-miR-582-5p |
| MPP1 | hsa-miR-592 |
| MPP1 | hsa-miR-607 |
| MPP1 | hsa-miR-886-3p |
| NOS3 | hsa-miR-886-3p |
| NOS3 | hsa-miR-155 |
| NOS3 | hsa-miR-220b |
| NOS3 | hsa-miR-31 |
| NOS3 | hsa-miR-362-5p |
| NOS3 | hsa-miR-492 |
| NOS3 | hsa-miR-500 |
| NOS3 | hsa-miR-502-5p |
| NOS3 | hsa-miR-506 |
| NOS3 | hsa-miR-524-3p |
| NOS3 | hsa-miR-543 |
| NOS3 | hsa-miR-576-3p |
| NOS3 | hsa-miR-744 |
| NPPA | hsa-miR-922 |
| NPPA | hsa-miR-105 |
| NPPA | hsa-miR-125a-3p |
| NPPA | hsa-miR-139-5p |
| NPPA | hsa-miR-194 |
| NPPA | hsa-miR-224 |
| NPPA | hsa-miR-425 |
| NPPA | hsa-miR-552 |
| NPPA | hsa-miR-576-3p |
| NPPA | hsa-miR-582-5p |
| NPPA | hsa-miR-607 |
| NPPA | hsa-miR-622 |
| NPPA | hsa-miR-802 |
| NPPB | hsa-miR-632 |
| NPPB | hsa-miR-21 |
| NPPB | hsa-miR-218 |
| NPPB | hsa-miR-220c |
| NPPB | hsa-miR-296-3p |
| NPPB | hsa-miR-374a |
| NPPB | hsa-miR-409-3p |
| NPPB | hsa-miR-617 |
| STUB1 | hsa-miR-922 |
| STUB1 | hsa-miR-198 |
| STUB1 | hsa-miR-212 |
| STUB1 | hsa-miR-324-3p |
| STUB1 | hsa-miR-329 |
| STUB1 | hsa-miR-331-3p |
| STUB1 | hsa-miR-455-3p |
| STUB1 | hsa-miR-545 |
| STUB1 | hsa-miR-608 |
| STUB1 | hsa-miR-625 |
| STUB1 | hsa-miR-634 |
| STUB1 | hsa-miR-873 |
| TFAM | hsa-miR-769-5p |
| TFAM | hsa-miR-299-5p |
| TFAM | hsa-miR-455-3p |
| TFAM | hsa-miR-556-5p |
| TFAM | hsa-miR-561 |
| TFAM | hsa-miR-582-3p |
| TFAM | hsa-miR-590-3p |
| TFB2M | hsa-miR-935 |
| TFB2M | hsa-miR-101 |
| TFB2M | hsa-miR-144 |
| TFB2M | hsa-miR-19a |
| TFB2M | hsa-miR-452 |
| TFB2M | hsa-miR-488 |
| TFB2M | hsa-miR-495 |
| TFB2M | hsa-miR-539 |
| TFB2M | hsa-miR-548c-3p |
| TIMP2 | hsa-miR-891a |
| TIMP2 | hsa-miR-130a |
| TIMP2 | hsa-miR-483-5p |
| TRAF2 | hsa-miR-767-3p |
| TRAF2 | hsa-miR-150 |
| TRAF2 | hsa-miR-188-3p |
| TRAF2 | hsa-miR-221 |
| TRAF2 | hsa-miR-342-3p |
| TRAF2 | hsa-miR-504 |
| TRAF2 | hsa-miR-532-3p |
| TRAF2 | hsa-miR-589 |
| TRAF2 | hsa-miR-601 |
| TRAF2 | hsa-miR-647 |
| UBQLN4 | hsa-miR-516b |
| UBQLN4 | hsa-miR-342-3p |

### REFERENCES

1. Mitchell P S, Parkin R K, Kroh E M, Fritz B R, Wyman S K, Pogosova-Agadjanyan E L, et al. Circulating microRNAs as stable blood-based markers for cancer detection. Proc Natl Acad Sci USA. 2008; 105:10513-10518.
2. Lopez-Romero P, Gonzalez M A, Callejas S, Dopazo A, Irizarry R A. Processing of Agilent microRNA array data. BMC Res Notes. 2010; 3:18.
3. Friedman R C, Farh K K, Burge C B, Bartel D P. Most mammalian mRNAs are conserved targets of microRNAs. Genome Res. 2009; 19:92-105.
4. Krek A, Grun D, Poy M N, Wolf R, Rosenberg L, Epstein E J, et al. Combinatorial microRNA target predictions. Nat Genet. 2005; 37:495-500.
5. Griffiths-Jones S, Grocock R J, van Dongen S, Bateman A, Enright A J. miRBase: microRNA sequences, targets and gene nomenclature. Nucleic Acids Res. 2006; 34:D140-144.
6. Huang da W, Sherman B T, Tan Q, Collins J R, Alvord W G, Roayaei J, et al. The DAVID Gene Functional Classification Tool: a novel biological module-centric algorithm to functionally analyze large gene lists. Genome Biol. 2007; 8:R183.
7. Aranda B, Achuthan P, Alam-Faruque Y, Armean I, Bridge A, Derow C, et al. The IntAct molecular interaction database in 2010. Nucleic Acids Res. 2010; 38:D525-531.
8. Salwinski L, Miller C S, Smith A J, Pettit F K, Bowie J U, Eisenberg D. The Database of Interacting Proteins: 2004 update. Nucleic Acids Res. 2004; 32:D449-451.
9. Ceol A, Chatr Aryamontri A, Licata L, Peluso D, Briganti L, Perfetto L, et al. MINT, the molecular interaction database: 2009 update. Nucleic Acids Res. 2010; 38:D532-539.
10. Cline M S, Smoot M, Cerami E, Kuchinsky A, Landys N, Workman C, et al. Integration of biological networks and gene expression data using Cytoscape. Nat Protoc. 2007; 2:2366-2382.
11. Goncalves J P, Graos M, Valente A X. POLAR MAPPER: a computational tool for integrated visualisation of protein interaction networks and mRNA expression data. J R Soc Interface. 2009; 6:881-896.

1. A biomarker panel comprising miR-16 (SEQ ID NO: 1), miR-27a (SEQ ID NO: 2), miR-101 (SEQ ID NO: 3), and miR-150 (SEQ ID NO: 4), for monitoring the prognosis of a patient having suffered from acute myocardial ischemia.

2. A biomarker panel according to claim 1 further comprising Nt-pro-BNP (SEQ ID NO: 5) for monitoring the prognosis of a patient having suffered from acute myocardial ischemia.

3. A method for monitoring the prognosis of a patient suffering from acute myocardial ischemia comprising analyzing a biomarker panel according to claim 1.

4. A method for predicting and/or monitoring the prognosis of left ventricular modeling in a patient, wherein the patient has suffered from an acute myocardial infarction, comprising determining the levels of miR-16, miR-27a, miR-101 and miR-150 in a sample of bodily fluid from said patient, and correlating the levels of said miRNAs with levels observed in a population of control patients who have not suffered from an AMI and have preserved left ventricular contractility, wherein a statistically significant increase in levels of miR-16 and mi-R27a and a statistically significant decrease in levels of miR-150 and miR-101 by comparison with the control is indicative of left ventricular contractility, or progress towards left ventricular contractility.

5. A method according to claim 4, wherein an increase in levels of Nt-pro-BNP by comparison with the control is also determined.

6. A method according to claim 4, wherein said patient has a WMIS score between 1 and 1.4.

7. A method for assessing the efficacy of a treatment for a patient having suffered from an acute myocardial infarction and having a likelihood of developing a reduced left ventricular contractility, wherein the method comprises i) determining the levels of miR-16, miR-27a, miR-101 and miR-150 in a sample of bodily fluid from said patient, ii) determining the Nt-pro-BNP level in a sample of bodily fluid from said patient, iii) determining the levels of miR-16, miR-27a, miR-101 and miR-150 and the level of Nt-pro-BNP in a sample of bodily fluid from said patient after treatment, iv) comparing the results of i) and ii) with the results of iii), wherein a difference between the results of i), ii) and iii) indicates an effect of the treatment.

8. A method according to claim 7, wherein said patient has a WMIS score between 1 and 1.4.

9. A method according to claim 4, wherein said body fluid is blood, serum, plasma, cerebrospinal fluid, saliva or urine, preferably blood, plasma or serum.

10. A diagnostic/prognostic kit for carrying out a method according to claim 4, comprising means for determining levels of miR-16, miR-27a, miR-101 and miR-150 in a sample of bodily fluid.

11. A composition comprising i) at least one short interfering nucleic acid capable of encoding a miRNA selected from the list consisting of miR-101 and miR-150 and at least one short interfering nucleic acid capable of inhibiting a miRNA selected from the list consisting of miR-16 and miR-27a or ii) short interfering nucleic acids capable of encoding miR-101 and miR-150 or iii) short interfering nucleic acids capable of inhibiting miR-16 and miR-27a for the treatment of left ventricular remodeling.

12. A pharmaceutical formulation comprising a composition of claim 11.

13. A model comprising establishing the levels of miR-16, miR-27a, miR-101 and miR-150 in a sample of bodily fluid from an MI patient, said model further comprising establishing the odds ratios of said miRNAs.

14. A model according to claim 13, further comprising establishing the level of Nt-pro-BNP and the associated odds ratio.

15. A model according to claim 13, wherein the probability P of developing left ventricular remodeling is calculated using the equation:
P=exp(X)/(1+exp(X));
wherein X=miR150×ln 0.08+miR101×ln 0.19+miR27a×ln 15.9+miR16×ln 4.18+Nt-pro-BNP×ln 3.97+territory×ln 2.29+STEM/NSTEMI×ln 1.68+Prior MI×ln 8.87+Hypercholesterolemia×ln 1.63+Hypertension×ln 1.00+Diabetes×ln 0.70+Smoking habit×ln 1.49+Gender×ln 1.29+Age×ln 1.00+ln 8.51×10E-5;
and wherein if P>0.5 then there is a significant risk of remodeling (WMIS>1.2);
and wherein if P<=0.5 then there is a low or null risk of remodeling (WMIS<=1.2).

16. A method according to claim 7, wherein said body fluid is blood, serum, plasma, cerebrospinal fluid, saliva or urine, preferably blood, plasma or serum.

17. A diagnostic/prognostic kit for carrying out a method according to claim 7, comprising means for determining levels of miR-16, miR-27a, miR-101 and miR-150 in a sample of bodily fluid.


SEQUENCE LISTING (5 sequences)

SEQ ID NO: 1 - DNA, 89 bp, Homo sapiens
  misc_feature: microRNA 16 (MIR16)
gtcagcagtgccttagcagcacgtaaatattggcgttaagattctaaaattatctccagtattaactgtgctgctgaagtaaggttgac

SEQ ID NO: 2 - DNA, 78 bp, Homo sapiens
  misc_feature: microRNA 27a (MIR27A)
ctgaggagcagggcttagctgcttgtgagcagggtccacaccaagtcgtgttcacagtggctaagttccgccccccag

SEQ ID NO: 3 - DNA, 75 bp, Homo sapiens
  misc_feature: microRNA 101 (MIR101)
tgccctggctcagttatcacagtgctgatgctgtctattctaaaggtacagtactgtgataactgaaggatggca

SEQ ID NO: 4 - DNA, 84 bp, Homo sapiens
  misc_feature: microRNA 150 (MIR150)
ctccccatggccctgtctcccaacccttgtaccagtgctgggctcagaccctggtacaggcctgggggacagggacctggggac

SEQ ID NO: 5 - protein, 134 aa, Homo sapiens
  misc_feature: natriuretic peptides B preproprotein
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH